(19) United States
(12) Patent Application Publication
(10) Pub. No.: US 2017/0273793 A1
(43) Pub. Date: Sep. 28, 2017

(54) ASSEMBLY OF LAYERED MONETITE-CHITOSAN NANOCOMPOSITE AND ITS TRANSITION TO ORGANIZED HYDROXYAPATITE

(71) Applicant: UNIVERSITY OF SOUTHERN CALIFORNIA, LOS ANGELES, CA (US)

(72) Inventors: JANET MORADIAN-OLDAK, LOS ANGELES, CA (US); QICHAO RUAN, ALHAMBRA, CA (US)

(21) Appl. No.: 15/466,221

(22) Filed: Mar. 22, 2017

Related U.S. Application Data
(60) Provisional application No. 62/311,626, filed on Mar. 22, 2016.

(51) Int. Cl. A61F 2/28; B32B 27/06; B32B 37/06

(52) U.S. Cl. CPC A61F 2/28; B32B 37/06; B32B 27/06; B32B 2309/105; B32B 2305/72; A61F 2002/30084

(57) ABSTRACT

A multilayer structure that functions as a bone replacement material includes a plurality of organic matrix layers such that each organic matrix layer includes chitosan and a dicarboxylic acid. The multilayer structure also includes a plurality of calcium phosphate-containing layers wherein each calcium phosphate-containing layer is interposed between a pair of organic matrix layers. Characteristically, the chitosan is cross-linked by the dicarboxylic acid.

### CROSS-REFERENCE TO RELATED APPLICATIONS

This application claims the benefit of U.S. provisional application Ser. No. 62/311,626 filed Mar. 22, 2016, the disclosure of which is hereby incorporated in its entirety by reference herein.

### STATEMENT REGARDING FEDERALLY SPONSORED RESEARCH OR DEVELOPMENT

The invention was made with Government support under Contract No. DE-020099 awarded by the National Institute of Dental and Craniofacial Research. The Government has certain rights to the invention.

### TECHNICAL FIELD

In at least one aspect, the present invention is related to compositions that can function as bone or enamel substitute materials.

### BACKGROUND

Calcium phosphates (CaP) are important biomaterials in the fields of tissue engineering and drug delivery, because of their low toxicity, excellent biocompatibility, and osteoconductivity. For example, nanostructured CaP materials have been used in biomedical products such as scaffolds for bone regrowth,¹ bioactive coatings and composites,² carriers for drug delivery,³ and bioactive fillers for the occlusion of exposed dentin tubules.⁴ However, the application of conventional CaP materials as hard tissue substitutes has been largely restricted by their brittleness and low strength. Progress to reinforce their mechanical properties through incorporation of high-strength materials in the CaP matrix has been incremental.^{5,6} Therefore, there is a need for an effective alternative strategy to boost the mechanical properties of synthetic CaP materials in a way to broaden their application in the areas of repair of multiple fractures of long bones, or vertebral body substitution.⁷

The hierarchical structure of biological composites has inspired scientists to develop high-performance materials with superior properties and functionalities.⁸⁻¹² Natural structural materials such as nacre, bone, and tooth exhibit excellent mechanical performance associated with their composition and unique hierarchical structures. For instance, the lamellar architectures in nacre and bone are well-known structures that provide these biocomposites with mechanical properties that far exceed those of their constituent materials.¹³ Another prime example, tooth enamel, consists of densely packed arrays of elongated apatite crystals organized into an intricate interwoven structure.¹⁴ Because of its unique architecture and dense mineral components, mature enamel possesses remarkably high elastic modulus and hardness, which are rarely achieved in artificial CaP materials.^{15,16} Thus, mimicking the structural features of such natural composites is a promising route to developing new structural materials with superior mechanical performance.^{13,16}

Over the past decade, efforts to synthesize CaP materials with bioinspired structures have relied on methods such as freeze casting,¹⁷ vacuum-assisted filtration,¹⁸ and biomimetic mineralization.^{19,20} In one such study, a bioinspired hydroxyapatite/poly(methyl methacrylate) composite with a nacre-mimetic lamellar structure was prepared by a bidirectional freezing method,¹⁷ and in another, Li et al. used a vacuum-assisted-filtration method to combine CaP plates with amyloid fibrils to generate a hybrid nanocomposite with bone-mimetic features.¹⁸ Bioinspired CaP materials with bone-mimetic or enamel-like structures have also been synthesized through matrix-regulating mineralization of apatite nanocrystals with the assistance of peptides,^{21,22} proteins,²³⁻²⁵ protein-inspired polymers,^{26,27} block copolymers,^{28,29} self-assembled liquid crystals,^{19,20} and other organic additives.^{30,31} These promising studies illustrate steps toward the formation of intricate structures at limited scale, although synthesis of bioinspired CaP materials with the architectural features over multiple length scales remains a great challenge. Another promising route to achieve hierarchical structures in CaP materials involves the use of a precursor crystal as a template. For example, based on the structural resemblance between monetite (dicalcium phosphate anhydrous, DCPA, CaHPO₄) and hydroxyapatite (HAp, Ca₅(PO₄)₃OH) crystals, monetite was shown to be an effective precursor in the preparation of various nanostructured hydroxyapatite crystals.³²⁻³⁴ Using macrosized crystals as a template, Liu et al. synthesized ordered HAp structures via a phase transformation from monetite.³⁵ This result demonstrated the feasibility of mimicking hierarchical designs of natural structures via a phase transformation from precursor crystals. However, the time-consuming preparation of the macro-sized monetite precursor (3 to 6 months) challenged its suitability for broader applications. Generating multilevel hierarchy is still a huge challenge in the fabrication of HAp from the direct transformation of a single monetite crystal.

Accordingly, there is a need for new materials that can functionally substitute for bone or enamel.

### SUMMARY

The present invention solves one or more problems of the prior art by providing in at least one embodiment, a method for forming a composite material. The method includes a step of dissolving chitosan, a dicarboxylic acid, a phosphate salt (e.g., NaH₂PO₄) and a calcium salt (CaCl₂.2H₂O) in water to form a reaction solution. The reaction solution is heated to form a layered monetite/chitosan/dicarboxylic acid composite (i.e., a first composite comprising monetite, chitosan, and a dicarboxylic acid). The layered monetite/chitosan/dicarboxylic acid composite is transformed into a calcium apatite/chitosan/dicarboxylic acid composite (i.e., a second composite comprising calcium apatite, chitosan, and a dicarboxylic acid).

In another embodiment, a composite formed by the method set forth herein is provided. Typically, this composite is a multilayer structure that functions as a bone or enamel replacement material. The multilayer structure includes a plurality of organic matrix layers and a plurality of calcium phosphate-containing layers. The organic matrix layer includes chitosan and a dicarboxylic acid. Characteristically, each calcium phosphate-containing layer is interposed between a pair of organic matrix layers.

### BRIEF DESCRIPTION OF THE DRAWINGS

FIG. 1A. Schematic of a multilayered composite formed by the methods of the invention.

FIG. 1B. Schematic representation of the pathway followed to fabricate multilevel organized CaP-based composite.

FIGS. 2A, 2B, 2C, 2D, 2E, and 2F. SEM images of (A, B) pure chitosan matrix and (C, D) organized chitosan-maleic acid (MAc) matrix: (C) top view, (D) side view. (E) XRD pattern of chitosan-MAc matrix. (F) Schematic representation of interaction between chitosan and MAc. The molecular model was established by ChemBio Ultra 11.0. The distance between the molecules was estimated using the typical bond lengths and angles. Blue in the chitosan chain and Red in the MAc molecule represent the charged amine and carboxyl groups, respectively.

FIGS. 3A, 3B, and 3C. (A, B) FTIR spectra of chitosan, MAc, and chitosan-MAc matrix in the absence of calcium in (A) the C═O stretch and (B) the N—H bending regions. (C) CD spectra of chitosan and chitosan-MAc matrix at pH 3.5.

FIGS. 4A, 4B, 4C, and 4D. (A, B) AFM and (C, D) TEM images of (A, C) pure chitosan and (B, D) organized chitosan-MAc matrix.

FIGS. 5A, 5B, 5C, 5D, 5E, 5F, and 5G. (A-C) SEM images of the orderly layered monetite-chitosan-MAc composite. (D) TEM and (E) HRTEM images of a monetite nanosheet. Inset: FFT pattern corresponding to e. (F) XRD pattern and (G) TG curve of the layered monetite-chitosan-MAc composite.

FIGS. 6A, 6B, 6C, and 6D. (A) pH values of reaction solution without (dotted line) and with (solid line) Ca²⁺ at different reaction times. (B) pH difference (ΔpH) between the solutions with and without Ca²⁺ at different time points. (C, D) SEM images of the products collected at (C) 1 and (D) 2 h.

FIGS. 7A and 7B. SEM images of the layered monetite-chitosan-MAc composites formed in chitosan-MAc matrix with different concentrations: (A) 0.5% (m/v) chitosan and 0.2% (m/v) maleic acid; and (B) 3% (m/v) chitosan and 1.2% (m/v) maleic acid.

FIG. 8. FTIR spectrum of the chitosan-MAc-Ca²⁺ complex.

FIGS. 9A, 9B, 9C, and 9D. Organized HAp-chitosan-MAc composite formed from phase transformation of layered monetite-chitosan-MAc composite. (A-C) SEM images of HAp-chitosan-MAc composite. Inset image in B shows the SEM image of an organized bundle. Arrows in (C) indicate different layers (stacks of HAp plates) in the HAp-chitosan-MAc composite. (D) HRTEM images of an isolated crystal from the HApchitosan-MAc composite. Inset: FFT pattern corresponding to D.

FIG. 10. Schematic representation of the hierarchical assembly process of organized chitosan-MAc matrix based on the data in FIGS. 2-4.

FIGS. 11A and 11B. (A) EDS and (B) FTIR spectra of the layered monetite-chitosan-MAc composite. The Ca/P was measured to be 1.0. The measured Ca/P molar ratio is 1.0, consistent with the stoichiometric ratio for monetite. The typical bands of monetite, as well as the characteristic peaks for chitosan and MAc, including δ(N—H) at 1645 cm⁻¹ for chitosan and ν(COO—) at 1555 cm⁻¹ for MAc, were found in the FTIR spectrum, showing that the precipitated monetite crystal platelets were covered with chitosan and MAc.

FIGS. 12A, 12B, and 12C. SEM images of the monetite crystals obtained (A) without organic matrix, (B) with chitosan alone, and (C) with maleic acid alone.

FIG. 13. Schematic representation of the formation of layered monetite-chitosan-MAc composite.

FIGS. 14A and 14b. (A) XRD and (B) EDS spectra of the organized HAp-chitosan-MAc composite. All of the diffraction peaks can be readily indexed to hexagonal phase hydroxyapatite (JCPDS 09-0432) crystals. EDS revealed the presence of calcium, phosphate, carbon and oxygen ions in the HAp-chitosan composite. The Ca/P molar ratio is measured to be 1.6, which is lower than the stoichiometric Ca/P ratio for hydroxyapatite, indicating an incomplete phase transformation consistent with HRTEM result in FIG. 9D.

FIGS. 15A and 15B. Crystal structures of (A) monetite and (B) hydroxyapatite. In monetite crystals, the CaHPO₄ lattice contains the Ca—PO₄ chains aligned in parallel to the b-axis, which are similar to those parallel to the c-axis in HAp. In addition, the length of b (6.62 nm) in CaHPO₄ lattice is roughly equal to that of c (6.68 nm) in HAp. As a result, the crystal topology structure of monetite is expected to be inherited by subsequent topotactic growth of HAp crystals under appropriate transition conditions.

FIG. 16. Representative photographic image of CaP tablet prepared from layered monetite-chitosan-MAc composite.

### DETAILED DESCRIPTION

Reference will now be made in detail to presently preferred compositions, embodiments and methods of the present invention, which constitute the best modes of practicing the invention presently known to the inventors. The Figures are not necessarily to scale. However, it is to be understood that the disclosed embodiments are merely exemplary of the invention that may be embodied in various and alternative forms. Therefore, specific details disclosed herein are not to be interpreted as limiting, but merely as a representative basis for any aspect of the invention and/or as a representative basis for teaching one skilled in the art to variously employ the present invention.

Except in the examples, or where otherwise expressly indicated, all numerical quantities in this description indicating amounts of material or conditions of reaction and/or use are to be understood as modified by the word “about” in describing the broadest scope of the invention. Practice within the numerical limits stated is generally preferred. Also, unless expressly stated to the contrary: percent, “parts of,” and ratio values are by weight; the term “polymer” includes “oligomer,” “copolymer,” “terpolymer,” and the like; molecular weights provided for any polymers refers to weight average molecular weight unless otherwise indicated; the description of a group or class of materials as suitable or preferred for a given purpose in connection with the invention implies that mixtures of any two or more of the members of the group or class are equally suitable or preferred; description of constituents in chemical terms refers to the constituents at the time of addition to any combination specified in the description, and does not necessarily preclude chemical interactions among the constituents of a mixture once mixed; the first definition of an acronym or other abbreviation applies to all subsequent uses herein of the same abbreviation and applies mutatis mutandis to normal grammatical variations of the initially defined abbreviation; and, unless expressly stated to the contrary, measurement of a property is determined by the same technique as previously or later referenced for the same property.

It is also to be understood that this invention is not limited to the specific embodiments and methods described below, as specific components and/or conditions may, of course, vary. Furthermore, the terminology used herein is used only for the purpose of describing particular embodiments of the present invention and is not intended to be limiting in any way.

It must also be noted that, as used in the specification and the appended claims, the singular form “a,” “an,” and “the” comprise plural referents unless the context clearly indicates otherwise. For example, reference to a component in the singular is intended to comprise a plurality of components.

The term “comprising” is synonymous with “including,” “having,” “containing,” or “characterized by.” These terms are inclusive and open-ended and do not exclude additional, unrecited elements or method steps.

The phrase “consisting of” excludes any element, step, or ingredient not specified in the claim. When this phrase appears in a clause of the body of a claim, rather than immediately following the preamble, it limits only the element set forth in that clause; other elements are not excluded from the claim as a whole.

The phrase “consisting essentially of” limits the scope of a claim to the specified materials or steps, plus those that do not materially affect the basic and novel characteristic(s) of the claimed subject matter.

With respect to the terms “comprising,” “consisting of,” and “consisting essentially of,” where one of these three terms is used herein, the presently disclosed and claimed subject matter can include the use of either of the other two terms.

Throughout this application, where publications are referenced, the disclosures of these publications in their entireties are hereby incorporated by reference into this application to more fully describe the state of the art to which this invention pertains.

### Abbreviations

“CaP” means calcium phosphates.

“CPC” means calcium phosphate cement.

“HAp” means hydroxyapatite.

“Hydroxyapatite” means calcium apatite usually approximated by the chemical formulae Ca₅(PO₄)₃(OH) or Ca₁₀(PO₄)₆(OH)₂.

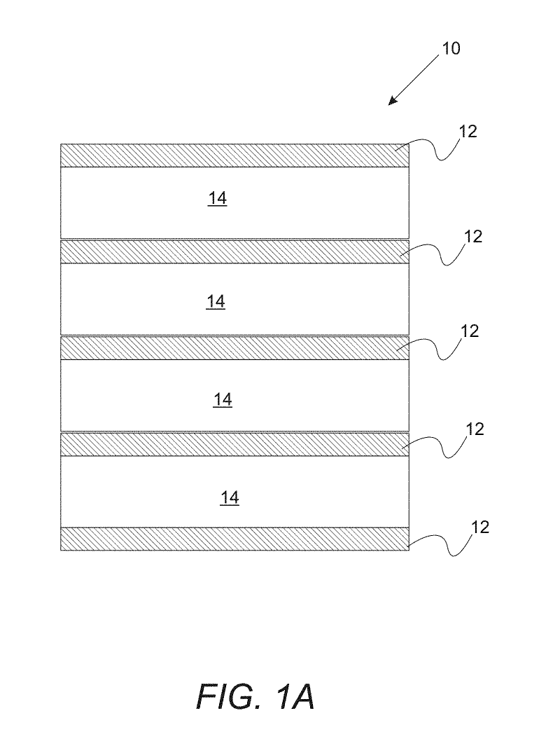

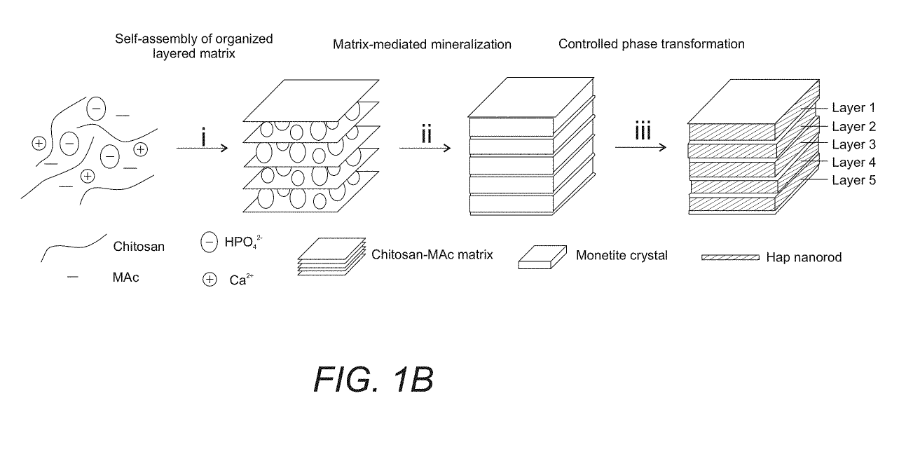

In at least one embodiment, a multilayer structure that functions as a bone replacement material is provided. With reference to FIG. 1A, multilayer structure 10 includes a plurality of organic matrix layers 12 such that each organic matrix layer includes chitosan and a dicarboxylic acid. The multilayer structure also includes a plurality of calcium phosphate-containing layers 14 wherein each calcium phosphate-containing layer is interposed between a pair of organic matrix layers. Characteristically, the chitosan is cross-linked by the dicarboxylic acid. Examples of suitable dicarboxylic acids include, but are not limited to, maleic acid, oxalic acid, fumaric acid, and combinations thereof. In certain variations, the calcium phosphate-containing layers are hydroxyapatite layers or monetite layers. In certain variations, the hydroxyapatite layers include HAp nanorods typically having a width from about 1 to 20 nm and a length from about 30 to 100 nm or more.

In some variations, the organic matrix layers have an average thickness greater than, in increasing order of preference, 10 nm, 20 nm, 30 nm, 50 nm or 100 nm. In another variation, the organic matrix layers have an average thickness less than, in increasing order of preference, 2 microns, 1 microns, 500 nm, 300 nm, 200 nm, or 100 nm. In a refinement, the organic matrix layers have an average thickness from about 50 nm to about 1 micron. In another refinement, the organic matrix layers have an average thickness from about 50 nm to about 200 nm. Similarly, in some variations, calcium phosphate-containing layers have an average thickness greater than, in increasing order of preference, 10 nm, 20 nm, 30 nm, 50 nm or 100 nm. In another variation, calcium phosphate-containing layers have an average thickness less than, in increasing order of preference, 2 microns, 1 microns, 500 nm, 300 nm, 200 nm, or 100 nm. In a refinement, the calcium phosphate-containing layers have an average thickness from about 50 nm to about 1 micron. In another refinement, the calcium phosphate-containing layers have an average thickness from about 50 nm to about 200 nm.

Another feature of the present invention is the number of layers in the multilayer structure. In a variation, the multilayer structure has, in increasing order of preference, greater than or equal to 2, 3, 4, 5, or 6 organic matrix layers. In a refinement, the multilayer structure has, in increasing order of preference, less than or equal to 50, 30, 10, 8, or 5 organic matrix layers. In a particular refinement, the multilayer structure has from 3 to 30 organic matrix layers and from 3 to 30 calcium phosphate-containing layers.

In another refinement, a method for making the multilayer structure set forth above is provided. The method includes a step of dissolving chitosan, a dicarboxylic acid, a phosphate salt (e.g., NaH₂PO₄) and a calcium salt (e.g., CaCl₂.2H₂O) in water to form a reaction solution. In a refinement, urea is included (i.e., added to) in the reaction solution. In a variation, the reaction solution includes 0.5 to 3 weight percent phosphate salt, 0.5 to 5 weight percent a calcium salt, 0.5 to 4 weight percent chitosan, and 0.1 to 1 weight percent of the C₂₋₆ dicarboxylic acid. In a refinement, the reaction solution includes 0.5 to 5 weight percent urea.

The reaction solution is heated to form a first composite which is a layered monetite/chitosan/dicarboxylic acid composite. As such, the first composite includes layered monetite, chitosan, and the dicarboxylic acid. The layered monetite/chitosan/dicarboxylic acid composite is then transformed into a second composite which is a calcium apatite/chitosan/dicarboxylic acid composite. In some variations, the second composite is a hierarchical composite with orderly bundle-like structures on the nanoscale (i.e., structures with dimensions of 1 to 100 nm) as well as layered organization on the microscale (e.g., layers organized with separations from 0.5 to 3 microns). As such, the second composite includes calcium apatite, chitosan, and the dicarboxylic acid. In one variation, the layered monetite/chitosan/dicarboxylic acid composite is contacted with base to form a calcium apatite/chitosan/dicarboxylic acid composite. As set forth above, examples of suitable dicarboxylic acids include, but are not limited to maleic acid, oxalic acid, fumaric acid, and combinations thereof. In a particularly useful refinement, the calcium apatite/chitosan/dicarboxylic acid composite is a hydroxylapatite/chitosan/maleic acid composite. Typically, chitosan in the themonetite/chitosan/dicarboxylic acid composite and/or the calcium apatite/chitosan/dicarboxylic acid composite (e.g., the hydroxylapatite/chitosan/maleic acid composite) is cross-linked by the dicarboxylic acid. Details regarding the number of layers in the formed multilayer structure are as the same as those set forth above.

As set forth above, the monetite/chitosan/dicarboxylic acid composite and/or the calcium apatite/chitosan/dicarboxylic acid composite (e.g., the hydroxylapatite/chitosan/maleic acid composite) can each independently be a multilayered structure having at least 3 organic matrix layers and at least 3 calcium apatite-containing layers. Typically, the organic matrix layers include the chitosan and the dicarboxylic acid. In a refinement, these composites can each independently be a multilayered structure having at least 5 organic matrix layers and at least 5 calcium apatite-containing layers. In another refinement, these composites can each independently be a multilayered structure having 3 to 30 organic matrix layers and 3 to 30 calcium apatite-containing layers. In a refinement, the organic matrix layers have an average thickness from about 50 nm to about 1 micron. In another refinement, the organic matrix layers have an average thickness from about 50 nm to about 200 nm.

The following examples illustrate the various embodiments of the present invention. Those skilled in the art will recognize many variations that are within the spirit of the present invention and scope of the claims.

In at least one aspect, the present invention provides a biomimetic strategy (inspired by the formation of brick-and-mortar structure in nacre) to generate a layered monetite-based composite that can further transform into a HAp-chitosan composite with a multilevel hierarchical structure. In nacre, the orderly layered structure forms under the mediation of an organic matrix composed of β-chitin, silk like proteins, and acidic glycoproteins rich in aspartic acid (Asp).³⁶⁻³⁸ Among these components, the oriented fibrils of β-chitin play an important role in regulating the crystal orientation, and the Asp-rich proteins are believed to interact both with the β-chitin substrate and the growing crystals.³⁶ Considering the critical functions of β-chitin and acidic proteins, our strategy starts with the rational design of an organic medium composed of chitosan and cis-butenediolic acid (maleic acid, MAc) to mimic the function of nacreous matrix and to generate an ordered layered structure. Chitosan is a deacetylated derivative of chitin usually used to study the crystallization process instead of chitin, owing to its soluble properties in acidic media. Experiments have shown that chitosan scaffolds are capable of promoting the formation of randomly sphere-like, rod-like, or plate-like CaP crystals,³⁹⁻⁴¹ yet no observable higher-order structure has been reported in these materials, perhaps because of the absence of orderly assembly of the pristine chitosan molecules.⁴² To overcome this problem, here we introduced a dicarboxylic MAc molecule that could serve as a cross-linking agent and lead to the intermolecular assembly of an organized matrix (FIG. 1, step (i)). In addition to the cross-linking capability, the carboxyl functional groups of MAc could also afford the matrix with interactive sites to nucleate the monetite crystals and mediate the formation of a layered structure (FIG. 1, step (ii)). Using this layered monetite precursor, we were able to synthesize a hierarchical composite with orderly bundlelike structures on the nanoscale as well as layered organization on the microscale through a controlled phase transformation process (FIG. 1, step (iii)). These hierarchical structures are expected to provide CaP materials with improved mechanical properties and potential for future hard tissue engineering applications.

### Materials and Methods.

Analyses of Chitosan-MAc Matrix. To investigate the assembly of the layered matrix, we prepared the chitosan-MAc solution by dissolving 0.25 g of chitosan (medium molecular weight, 75-85% deacetylated, Sigma-Aldrich) and 0.1 g of maleic acid (MAc, Sigma-Aldrich) in 25 mL of deionized water containing 0.72 mL of acetic acid under magnetic stirring, and the pH value was adjusted to 3.5 with 1 M NaOH. The solid phase of chitosan-MAc matrix was obtained by lyophilization for scanning electron microscopy (SEM), X-ray diffraction (XRD), and Fourier transform infrared spectroscopy (FTIR) studies. For circular dichroism (CD), atomic force microscopy (AFM) and transmission electron microscopy (TEM) studies, 0.1 mL of chitosan-MAc solution was diluted with 0.9 mL deionized water. The TEM sample was prepared by depositing a droplet of the diluted chitosan-MAc solution onto a TEM grid and then staining it with a droplet of a 0.1 wt % phosphotungstic acid aqueous solution.

Preparation of Layered Monetite-Chitosan-MAc Composite. Six grams of urea, 0.25 g of chitosan (medium molecular weight, 75-85% deacetylated, Sigma-Aldrich) and 0.1 g of maleic acid (MAc, Sigma-Aldrich) were dissolved in 25 mL of deionized water containing 0.72 mL of acetic acid under magnetic stirring. Then, 0.3 g of NaH₂PO₄ and 0.614 g of CaCl₂.2H₂O were dissolved into the solution under vigorous stirring for 2 h, and the pH value was adjusted to 3.5 with 1 M NaOH. Next, the reaction solution was heated in a water bath at 70° C. for 4.5 h, then allowed to cool down to room temperature naturally. Following the reaction, the obtained suspension was centrifuged and washed with distilled water and anhydrous ethanol several times. Finally, the sample was frozen on dry ice and lyophilized for 16 h.

Preparation of Organized HAp-chitosan-MAc Composite via Phase Transition. Layered monetite-chitosan-MAc composite (0.15 g) was dispersed in 15 mL of NaOH solution (0.1 M, pH 13) and kept in a water bath under vigorous stirring at 70° C. for 5 min. The suspension was then immediately centrifuged and washed with distilled water and anhydrous ethanol several times. Finally, the sample was frozen on dry ice and lyophilized overnight.

Preparation of Calcium Phosphate Tablets. The CaP tablets were prepared by pressing the layered monetite-chitosan-MAc or organized HAp-chitosan-MAc composite in a stainless steel mold under a pressure of 10 MPa at 130° C. for 48 h.

Characterization. SEM imaging was performed in a JSM-7001F field emission scanning electron microscope (JEOL, Peabody, Mass.) operating at an accelerating voltage of 5-15 kV. XRD patterns were recorded by a Rigaku diffractometer with Cu Kα radiation (λ=1.542 Å) operating at 70 kV and 50 mA with a step size of 0.02°, at a scanning rate of 0.1°/sec in the 20 range 10-60°. TEM images were obtained on a JEOL JEM-2100 microscope using an accelerating voltage of 200 kV. CD spectropolarimetry was performed using a J-815 spectropolarimeter (JASCO, Easton, Md.). The spectra were recorded between 190 and 260 nm with a step size of 0.5 nm and a scan rate of 50 nm/min. AFM images were obtained by using a NanoScopeIIIa scanning probe microscope system (Digital Instruments, Tonawanda, N.Y.) with a tapping mode etched silicon probe (model OTESPA-10) in air at scanning rates of 0.5-1.0 Hz for different resolutions. FTIR spectra were acquired from a Nicolet 4700 Spectrometer with a GladiATR diamond crystal accessory. Thermogravimetry (TGA) analysis was performed on a Q5000 IR (TA Instruments, New Castle, Del.) with a heating rate of 50° C./min, under an air flow of 20 mL/min from 30 to 900° C. The hardness and elastic modulus were measured at 25 test points in each sample (n=3) using a nanoindenter (AgilentMTS XP) with a Berkovich tip.

Statistical Analysis. The experiments were conducted in triplicate and the data were expressed as mean±standard deviations. Student's t test was applied to identify differences in the hardness and elastic modulus between layered monetite-based composite and hierarchical HAp-based composite. The differences were considered statistically significant at p<0.05 and highly significant at p<0.001. All the statistical analyses were carried out using Origin 8.0 (Origin lab, Northampton, Mass.) and Microsoft Office Excel 2007.

Results and Discussion

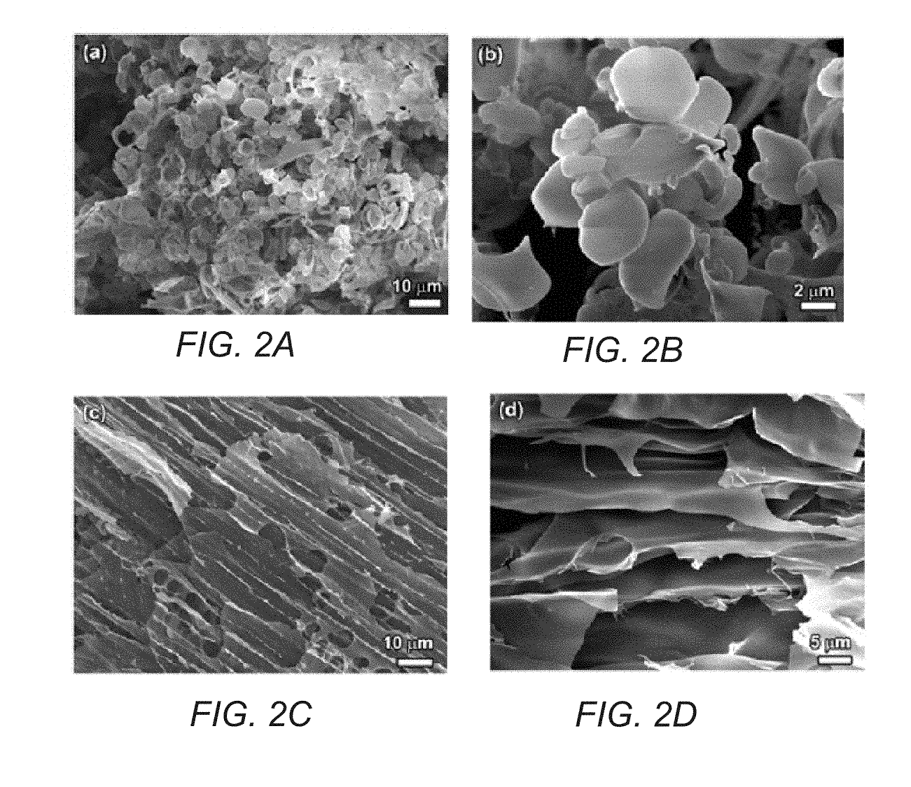

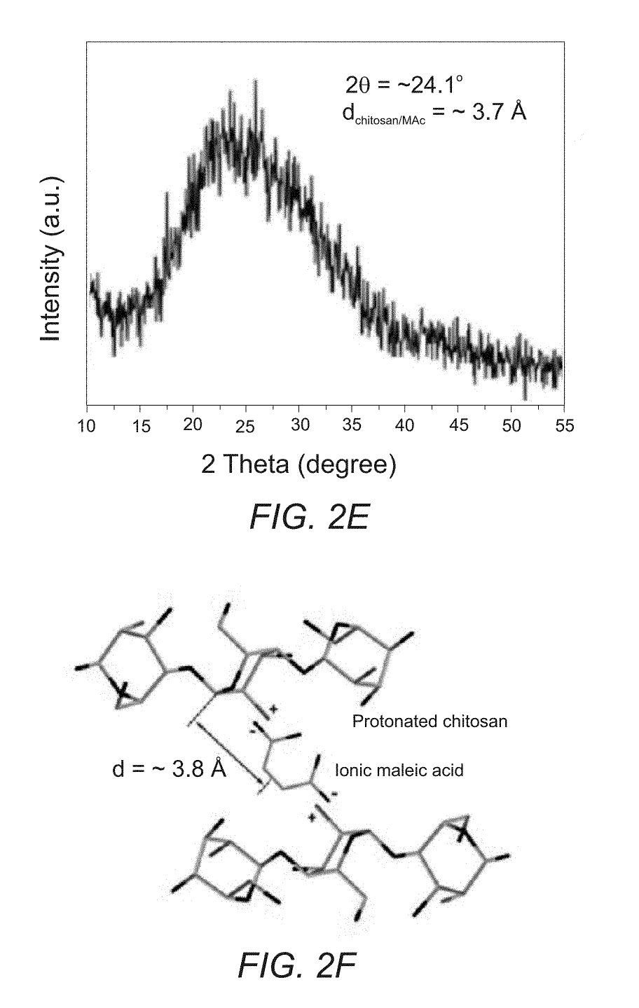

Assembly of Organized Chitosan-MAc Matrix. Unlike the chitin in nacre matrix, chitosan alone does not form an organized structure because it lacks the acetylamide groups that contribute to hydrogen bonding during the self-assembly of chitin.⁴² In our study, only irregularly spherical-like morphology can be observed when examining pure chitosan (FIG. 2a, b). Inspired by the critical role of intermolecular interaction in chitin assembly, we included maleic acid (MAc) into the chitosan matrix to provide an interactive cross-linker and facilitate the orderly assembly of the organic matrix.

FIGS. 2C, D show the sheet like morphology of the organic matrix composed of chitosan and MAc. These chitosan-MAc sheets are stacked in parallel layered structures similar to the organized structures seen in nacreous matrix.⁴³ In addition, the XRD pattern of chitosan-MAc matrix displays a diffraction peak at a spacing of 0.37 nm (FIG. 2E), representing short-range order in the chitosan-MAc molecular complex (FIG. 2F).

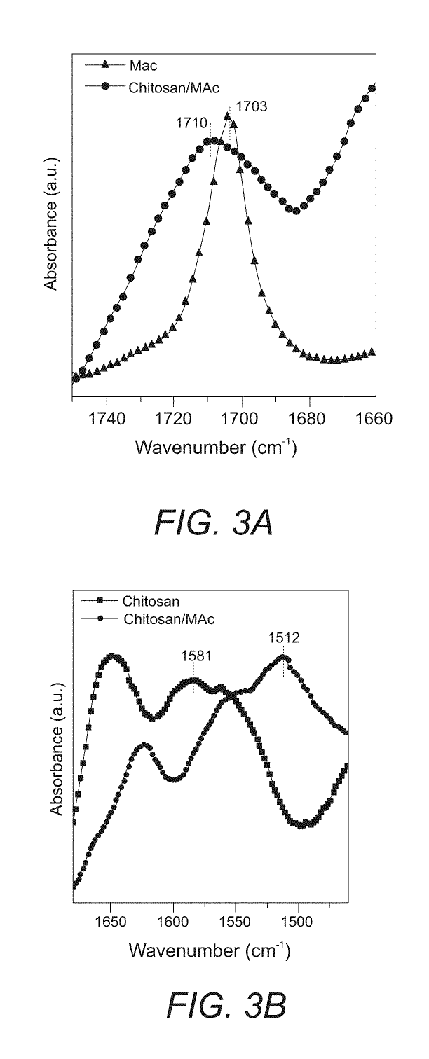

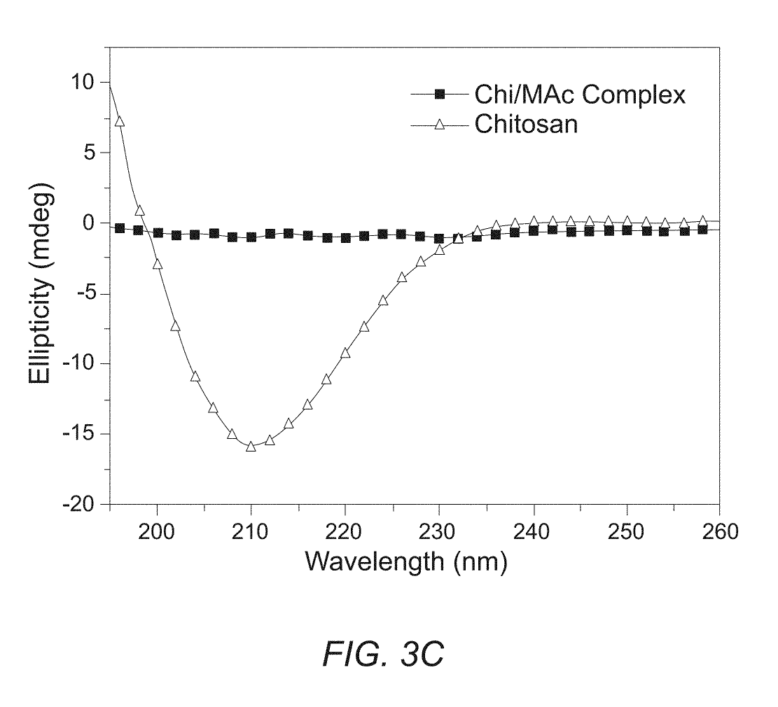

These observations demonstrate the ordered coassembly of chitosan-MAc matrix due to the specific interaction between chitosan and MAc, which was further confirmed by FTIR. Compared with the FTIR spectra of pure chitosan or MAc, the COO— stretch vibration band shifted from 1703 to 1710 cm⁻¹ in the spectra of chitosan-MAc mixture (FIG. 3a), and the N—H bending vibration band shifted from 1581 to 1512 cm⁻¹ (FIG. 3b).

These shifts indicate a strong interaction between the charged carboxyl and amine groups.^{42,44,45} Indeed, under our experimental conditions (3.5<pH<4.5), the amine groups of chitosan (pKₐ=6.5) are protonated to form positively charged chains.⁴⁶ Simultaneously, the two carboxyl ends of MAc (pKₐ=1.92) are fully ionized.⁴⁷ As a result, the chitosan chains can be cross-linked by MAc molecules through hydrogen bonding and electrostatic interactions between the positively charged amine groups and the negatively charged carboxyl groups (FIG. 2f). The specific chitosan-MAc interaction is expected to cause a conformational change in the chitosan molecule, which is confirmed by the circular dichroism (CD) spectropolarimetry. As shown in FIG. 3c, the CD spectrum of pure chitosan in aqueous solution (pH 3.5) has a broad negative CD band located at about 210 nm, corresponding to the n→π* electronic transition of the —NH—CO-chromophore of GlcNAc (N-acetyl-D-glucosamine) units.⁴⁸ After adding MAc to the chitosan solution, the negative dichroic signal of chitosan is absent (FIG. 3C), indicating a distinct change in the chromophore environments of the GlcNAc units due to the interaction between chitosan and MAc.⁴⁹ This conformational change may give rise to an anisotropic ligand distribution in the chitosan-MAc complex that could further assist in the assembly of an organized matrix.^{50,51}

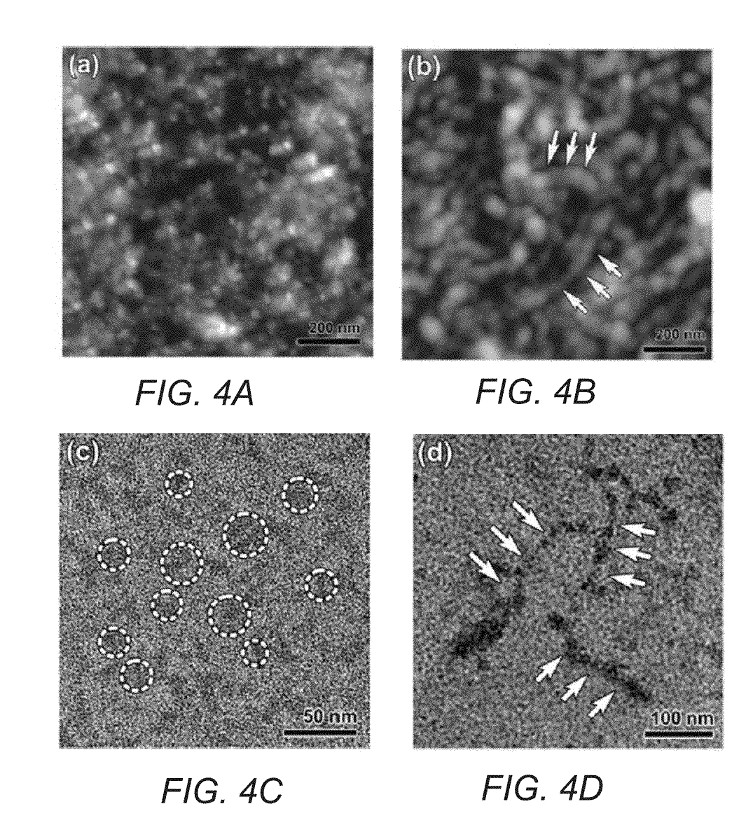

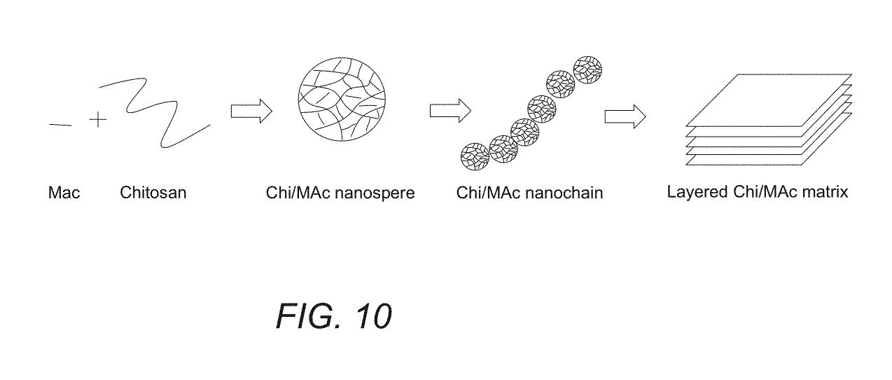

The assembly states of pure chitosan and chitosan-MAc matrix were revealed by tapping mode atomic force microscopy (AFM) and transmission electron microscopy (TEM) under the conditions described in FIG. 3. Without MAc, the chitosan chains tend to aggregate into randomly distributed nanoparticles with a size of ˜30 nm due to hydrophobic or solvophobic interactions (FIG. 4A, B), as proposed elsewhere.⁵²⁻⁵⁵ In contrast, following addition of MAc we could observe linear chains of ˜50 nm nanoclusters in the chitosan-MAc matrix (arrows in FIG. 4B, D). We speculate that this spontaneous linear assembly depends on an equilibrium between various attractive and repulsive interactions, such as hydrophobic interaction, van der Waal's attraction, hydrogen bonding and electrostatic repulsion.^{56,57} The attractive forces favor more contact between nanoparticles and drive the formation of compact aggregates, although electrostatic repulsion acts as a key driving force to form anisotropic self assemblies of 1D chains.⁵⁸ The insertion of ionized MAc molecules into a chitosan system may dramatically disrupt the hydrophobic force within a close distance.⁵⁸ As a result, the repulsion between neighboring chitosan-MAc nanoparticles tends to force these molecules into an ordered assembly to evenly distribute their charges. This process eventually leads to the formation of chitosan-MAc sheets that further stack into a layered structure. Based on the results discussed here (FIGS. 2-4), the self-assembly process of layered chitosan-MAc matrix is schematically presented in FIG. 10. Using this chitosan-MAc matrix, we were able to further construct an orderly, layered monetite composite.

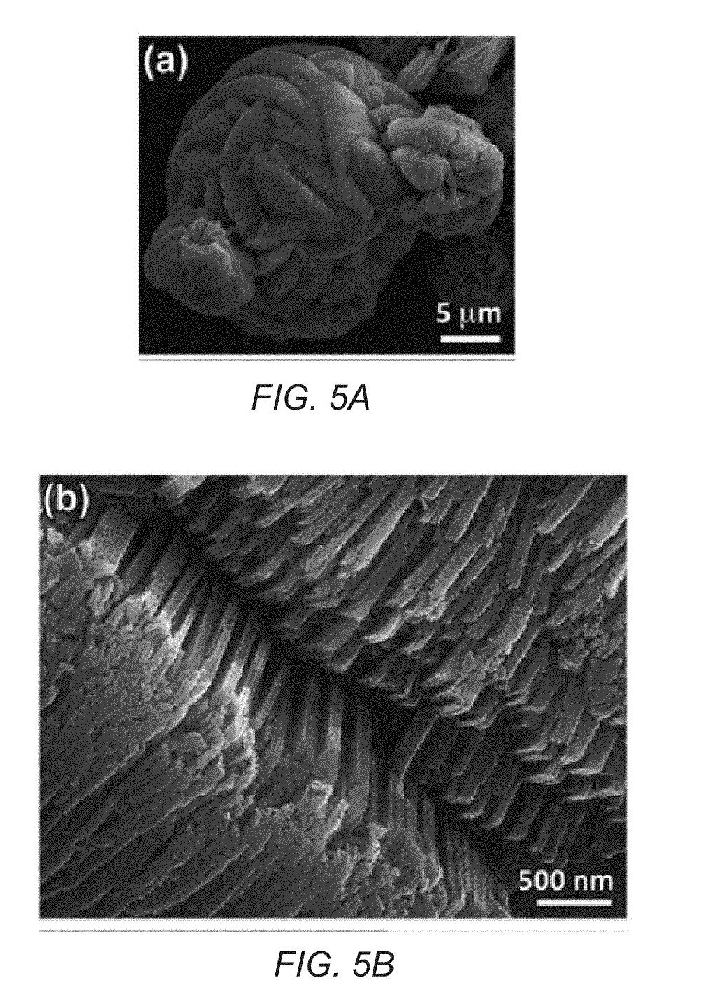

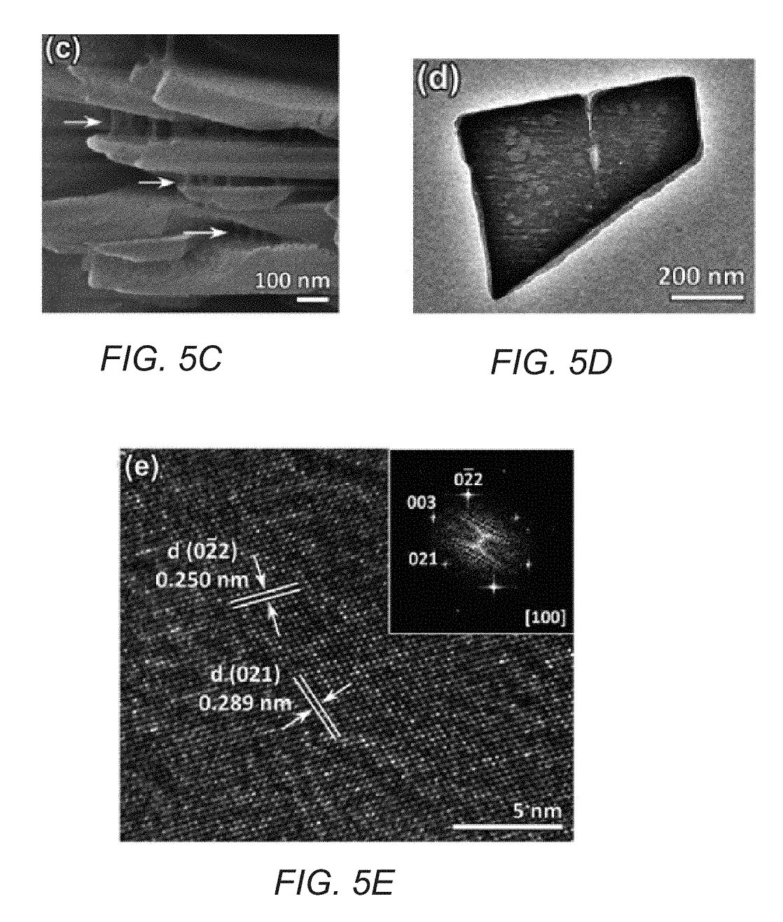

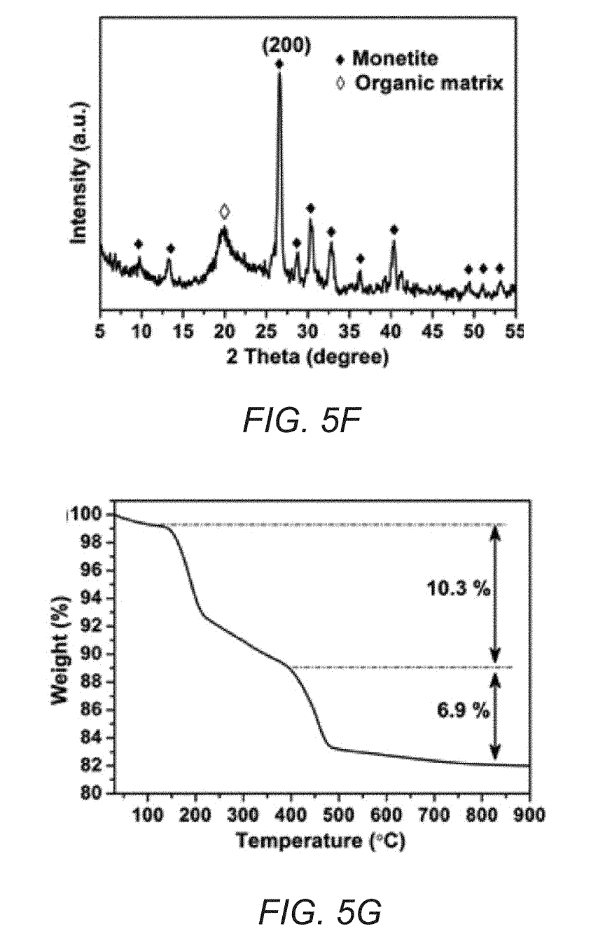

Assembly of Layered Monetite-Chitosan-MAc Composite. To prepare a calcium phosphate composite with an orderly layered structure, we adopted a wet-chemical precipitation method in the presence of a chitosan-MAc mixture. The resulting calcium phosphate products exhibited a brick-and-mortar structure (FIG. 5A-D), in which calcium phosphate platelets with a thickness of ˜100 nm were bound together by the organic binder (arrows in FIG. 5C). Highresolution transmission electron microscopy (HRTEM) and corresponding fast Fourier transform (FFT) images provide further insight into the structural details of the calcium phosphate platelets (FIG. 5E). The interplanar distances measured in segments of HRTEM micrograph were 0.250 and 0.289 nm, corresponding to the (022) and (021) planes of monetite crystals (JCPDS Card: 71-1759), respectively. The corresponding FFT pattern further confirms that the calcium phosphate platelet has a monetite polymorph (inset in FIG. 5e) imaged along the [200] direction, indicating that the facet planes of the monetite platelets are (200).

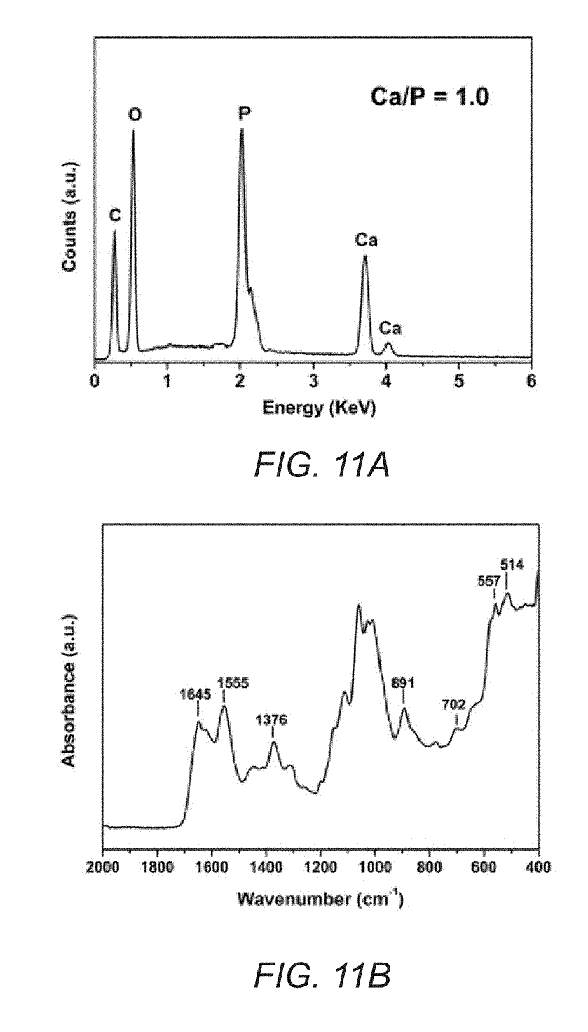

This crystallographic characterization and composition of the layered composite were further confirmed by XRD and energy dispersive X-ray spectroscopy (EDS) (FIG. 5F and FIG. 11A). The resulting diffractogram shows that the products are mainly composed of monetite crystals (JCPDS Card: 71-1759). A strong 200 diffraction peak indicates a preferred (200) orientation of the monetite platelets in accordance with the above HRTEM analysis. In addition, a broad diffraction peak around 2θ=20° may be ascribed to the presence of organic matrix in the layered composite. EDS reveals the presence of calcium, phosphorus, carbon, and oxygen in the layered composite. The measured Ca/P molar ratio is 1.0, consistent with the stoichiometric ratio for monetite. The composite nature of the material is also revealed by FTIR spectroscopy and thermogravimetric analysis (TGA). For example, the typical bands of monetite, as well as the characteristic peaks for chitosan and MAc, including δ(N—H) at 1645 cm⁻¹ for chitosan⁴⁴ and ν(COO—) at 1555 cm⁻¹ for MAc,⁵⁹ are found in the FTIR spectrum, indicating that the precipitated monetite crystal platelets are covered with chitosan and MAc (FIG. 11B). The TG curve in FIG. 5G shows that the total weight loss of monetite-chitosan composite is 17.2% in the range of 100-900° C., whereas pure monetite should give a weight loss of 6.6% when it is decomposed completely (2CaHPO₄ [symbol] Ca₂P₂O₇+H₂O).⁶⁰ Aside from the weight loss caused by the decomposition of monetite, the additional weight loss of ˜10.6% corresponds to loss of water and the organic component. The structural and compositional analyses indicate that the products synthesized in the chitosan-MAc matrix contain monetite with an organized, layered arrangement.

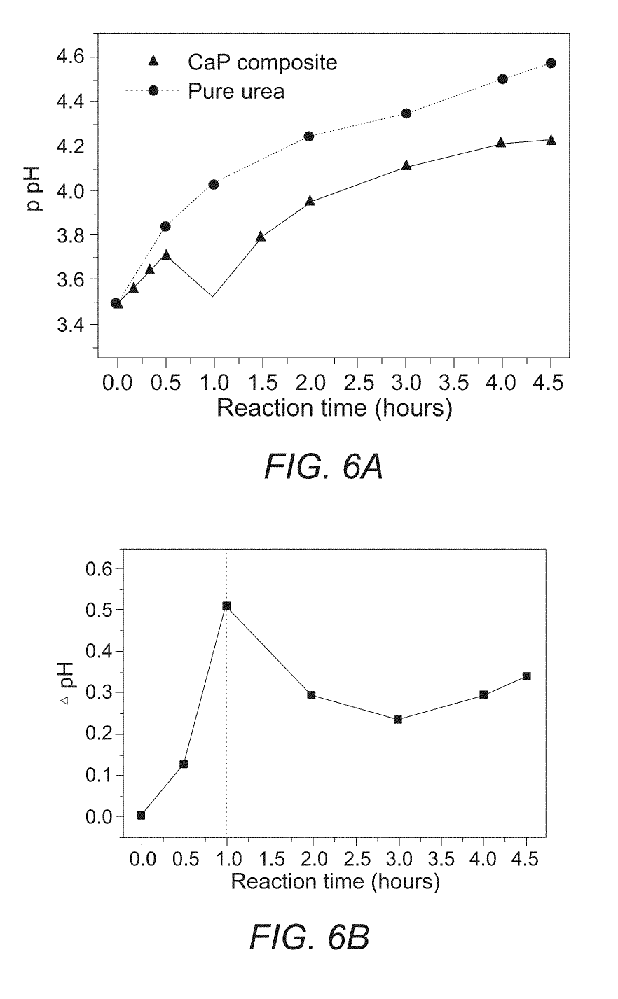

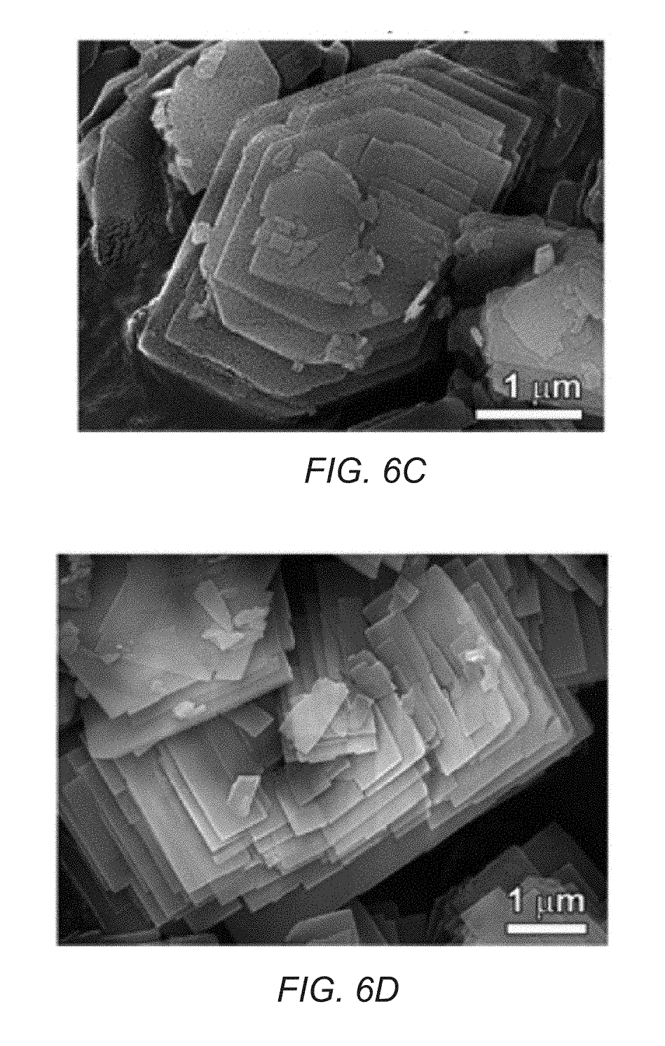

To investigate the assembly process of the layered monetite-chitosan-MAc composite, the pH of the reaction system and the product morphology were monitored at different reaction times (FIG. 6). When the solution contained no Ca²⁺, the pH increased with prolonged reaction time due to the hydrolysis of urea (dotted line in FIG. 6A). In contrast, a reduction in pH was observed in the reaction solution when the reaction time increased to 1 h (solid line in FIG. 6A). Moreover, the addition of Ca²⁺ in the reaction solution resulted in lower pH values because of the nucleation of monetite in the system. To diminish the effect of urea hydrolysis on the pH change and better understand the crystallization process, the pH difference (ΔpH) between the solutions with and without Ca²⁺ was calculated at different reaction times. As shown in FIG. 6b, the ΔpH rose from 0 to 0.5 within the first hour of reaction and dropped to ˜0.3 when reaction time reached 2 h. After that, only slight changes were seen in the ΔpH. On the basis of these observations, the possible reaction process in the chitosan-MAc matrix can be inferred as follows (eqs 1-3).

CO(NH)₃₂+2HO₂ [symbol] 2NH₄+2OH+CO₂ (1)

HPO_{2→4}⁻+OH⁻→HPO₄²+H₂O (2)

Ca²⁺+HPO₄²⁻→CaHPO₄ (3)

H₂PO₄⁻ is dominant in the initial reaction solution with a pH of 3.5. As reaction time increases, the pH gradually increases due to the hydrolysis of urea (eq 1), leading to a conversion of H₂PO₄⁻ to HPO₄²⁻ (eq 2).⁶¹ When the concentration product of Ca²⁺ and HPO₄²⁻ is larger than the Ksp of monetite, the monetite starts crystallizing in the solution (eq 3). Meanwhile, the nucleation of crystals leads to a decrease in the pH of the reaction solution because of the consumption of Ca²⁺ and OH⁻ (eqs 2 and 3). When the reaction time is increased to 1 h, white precipitation of monetite is formed in the solution. The products obtained at this time consist of monetite platelets assembled in an orderly layered structure (FIG. 6C). After 2 h, there are no significant changes in ΔpH, indicating that the main reaction in the solution is the assembly of monetite crystals. At this stage, the initially formed crystals (FIG. 6C) further stack and aggregate together (FIG. 6D) and eventually form ˜30 μm spheres, as shown in FIG. 5A.

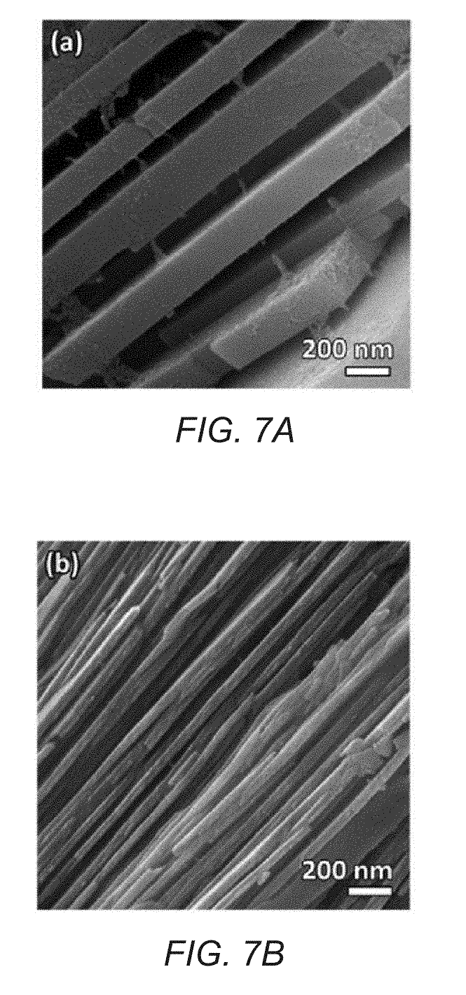

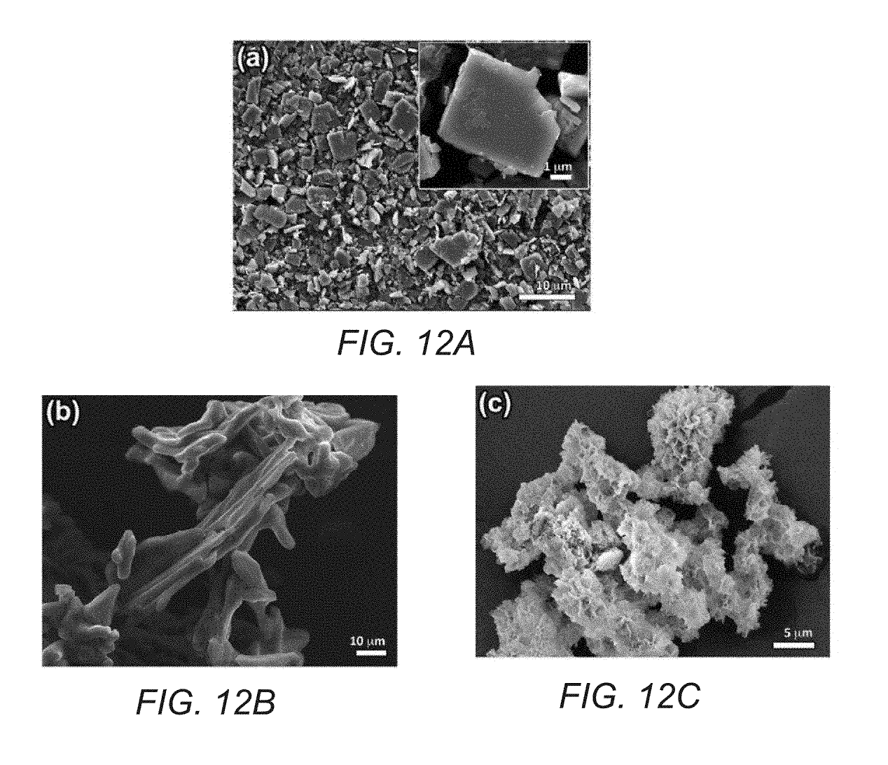

We can further consider the role of the chitosan-MAc mixture in this assembly mechanism. We did not observe any layered structure at any point during the reaction process in the absence of either chitosan or MAc. In a comparative experiment without chitosan and MAc, the monetite produced from precipitation formed plate-shaped structures with a width of 5-10 μm (FIG. 12A). Only platy monetite crystals were formed in the absence of MAc (FIG. 12B), and only porous HAp was produced in the absence of chitosan (FIG. 12C). Other evidence for the critical role of the chitosan-MAc matrix is provided in a series of experiments in which we examined the thickness of monetite crystals as a function of chitosan and MAc concentration. Remarkably, changes in the thickness of monetite crystals were observed upon altering the concentration of chitosan-MAc matrix in a dose dependent manner. As shown in FIGS. 7 and 5b, the thickness of monetite crystals decreased from ˜200 nm to ˜100 nm and ˜50 nm when the concentration of chitosan increased from 0.5% (m/v) to 1% (m/v) and 3% (m/v). (Note: the mass ratio of chitosan to maleic acid was kept steady at 2.5.) These observations suggest that the chitosan-MAc mixture is crucial to the construction of orderly layered monetite composite.

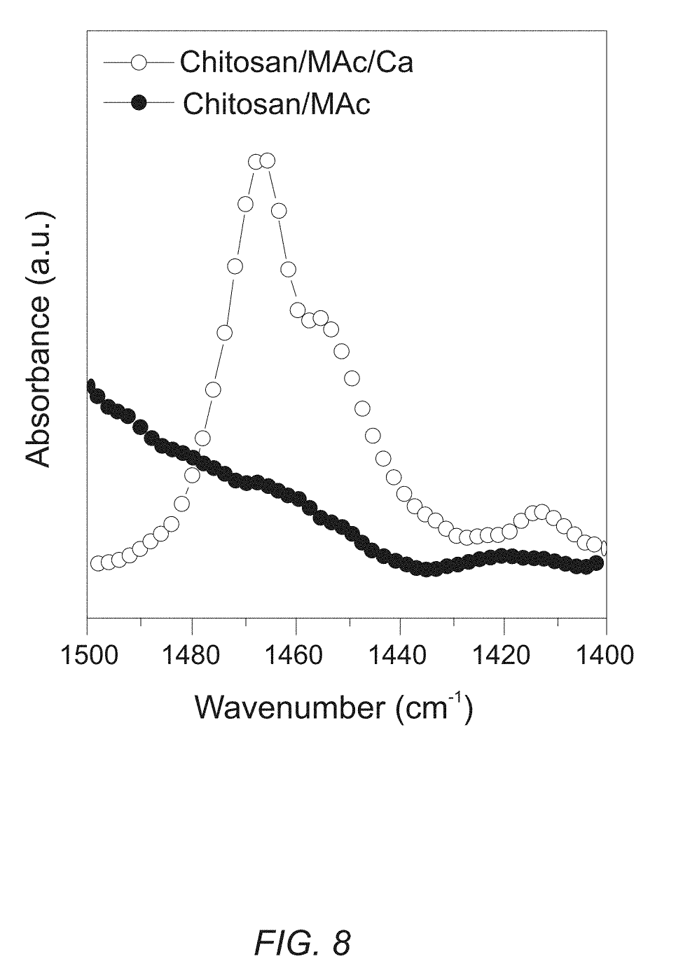

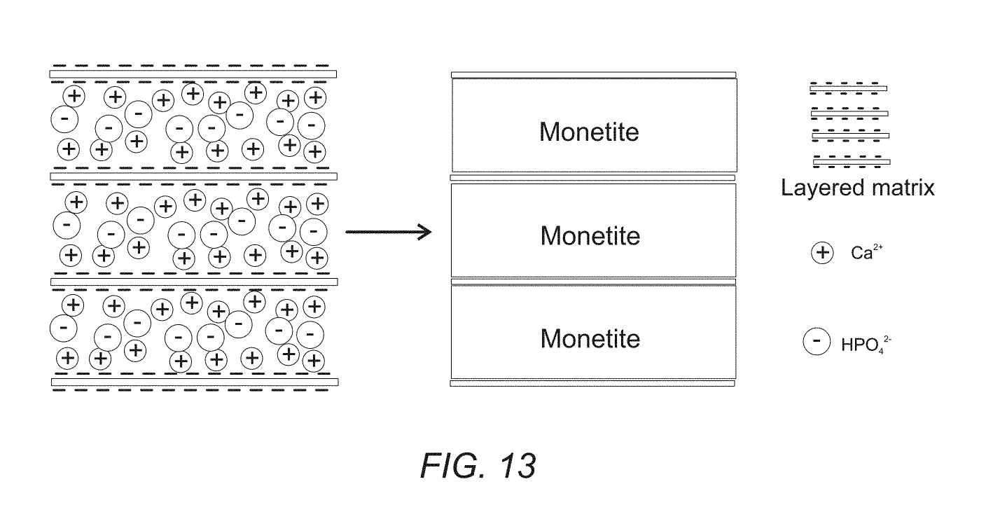

It is noteworthy that the pH values (3.5<pH<4.5) of the reaction solution are in between the pKₐ values of maleic acid (1.92) and chitosan (6.5) throughout the reaction process. As a result, the protonated amine groups and ionized carboxyl groups remain active and interact with each other, leading to a persistent assembly and function of the chitosan-MAc matrix during the formation of layered monetite composite. Similar to the formation of nacre,^{37,38} the chitosan-MAc layers not only function as a structural framework, but also provide a charged carboxylate surface that plays an important role in inducing nucleation and interacting with forming crystals. The negatively charged carboxyl groups of the chitosan-MAc complex are preferentially adsorbed on the calcium-rich (100) surface of crystals to stabilize and confine the formation of monetite platelets. The interaction between the organic complex and the calcium ions can be verified by the appearance of a characteristic FTIR vibration of COO—Ca at 1466 cm⁻¹ (FIG. 8).³² The proposed formation process of layered monetite-chitosan-MAc composite under the mediation of chitosan-MAc matrix is summarized as follows (FIG. 13). First, the negatively charged carboxyl groups of chitosan-MAc matrix act as active sites to interact with calcium and phosphate ions, inducing the heterogeneous nucleation of monetite. Then, guided by the chitosan-MAc matrix template, the monetite platelets are formed in between the layers of the chitosan-MAc scaffold, and finally grow in an organized manner into an orderly layered structure.

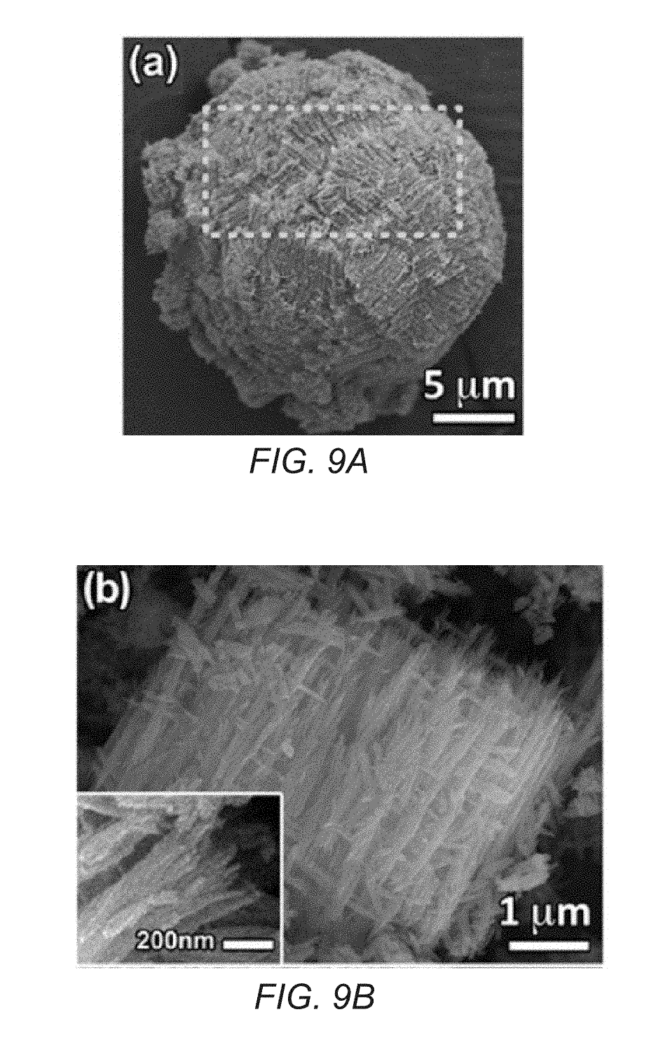

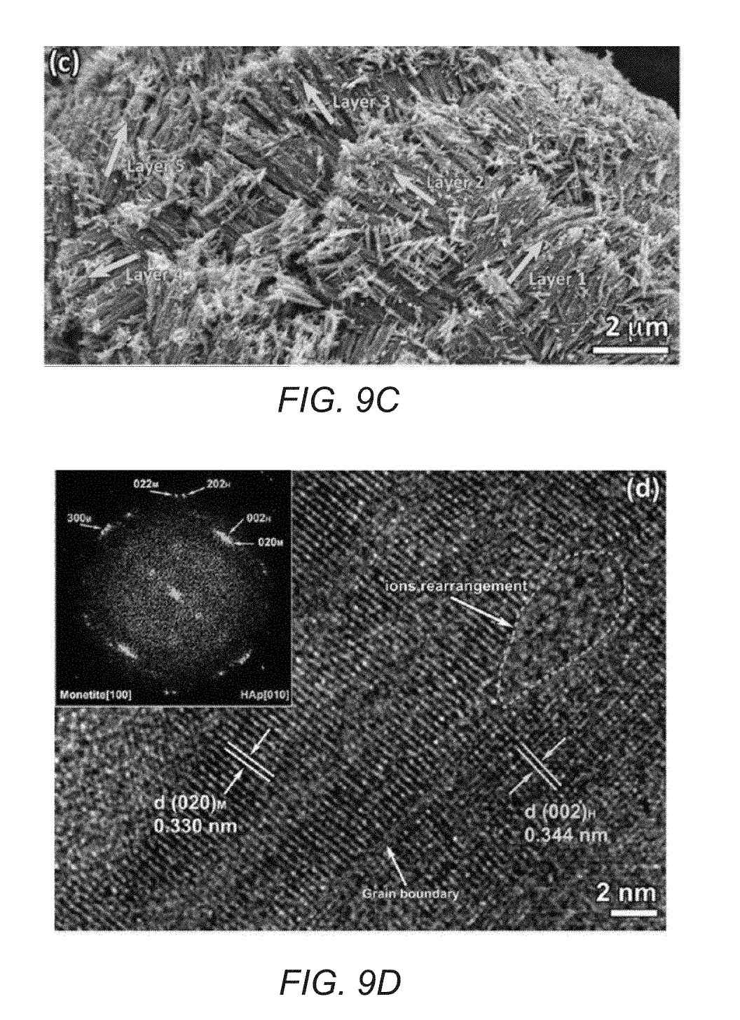

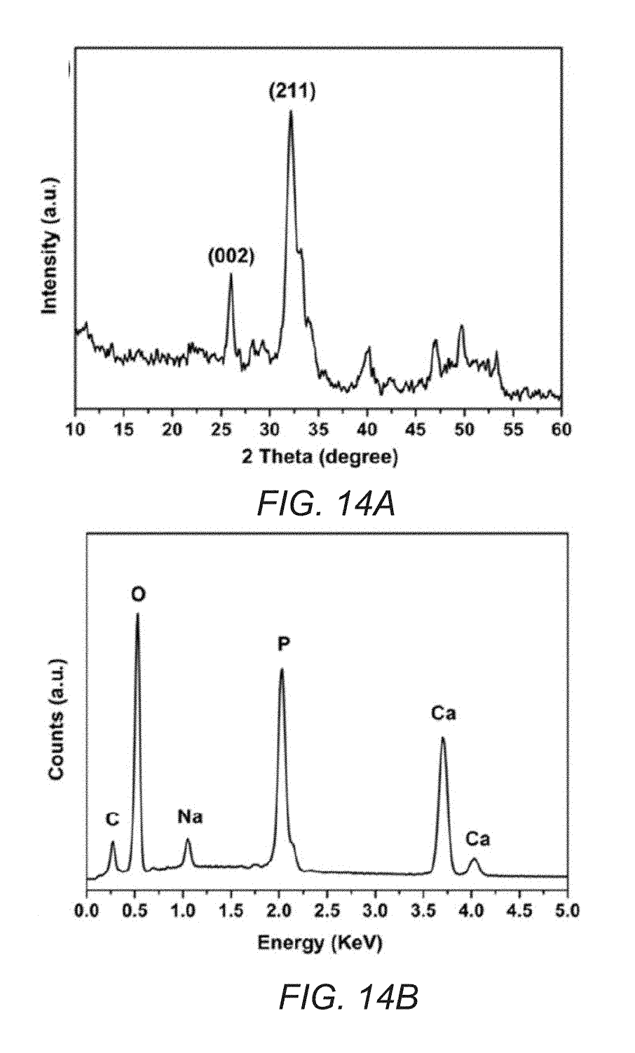

Synthesis of Organized HAp-Chitosan-MAc Composite via Phase Transition. Using the layered monetitechitosan-MAc composite, we were able to fabricate an organized HAp-chitosan-MAc composite via a phase transformation in an alkali solution at 70° C. XRD and EDS results confirmed that hydroxyapatite (JSPDS Card: 09-0432) was obtained after immersing the monetite-chitosan composite in a 0.1 M NaOH (pH 13) solution for 5 min (FIG. 14). This process produced a multilevel ordered structure, as shown in FIG. 9A-C. On the nanoscale, needlelike HAp crystallites with a diameter of ˜25 nm assembled into organized bundles (inset in FIG. 9b). On the microscale, these HAp bundles aligned to form highly oriented plates about 2 μm in width and 4 μm in length (FIG. 9B). Most interestingly, on the larger-scale level, these HAp plates with different crystal orientations stacked to form a layered structure (arrows in FIG. 9C). A typical layer consisted of a HAp plate with an estimated thickness of ˜100 nm, which was inherited from the monetite precursor. Clearly, the layered arrangement of the monetite precursor was preserved to some extent during the phase transformation.

During the phase transformation from monetite to HAp, the ordered nanostructure can only be obtained through a topotactic solid-solid transition under highly alkaline conditions (10CaHPO₄+6OH⁻→Ca₁₀(PO₄)₆(OH)₂+4PO³₄⁻+10H₂O).³⁵ Specifically, the highly alkaline conditions generate a strong driving force that compels the ions to move and rearrange inside the crystal, resulting in a spatially periodic nucleation of HAp seeds and formation of well-arranged HApnanoneedles on the monetite surface. However, the structural stress induced by dehydration and the movement of ions during the transition from monetite to HAp can cause changes to the crystal morphology and collapse of the layered structures.^{34,62} Thus, a major challenge associated with synthesis of hierarchical HAp in the present study was to preserve the layered arrangement on a larger-scale level during the phase transformation from monetite composite to HAp. One way to overcome this problem is to use an organic matrix that can protect the organized layered structures. For example, by using different biomacromolecules and surfactants, the crystal morphology can be maintained during the phase transformation from OCP to HAP because of the interaction between organic molecules and crystals.⁶¹ In a similar way, the chitosan-MAc matrix was expected to preserve the hierarchical architecture of crystals during the monetite-HAp transition by interacting with the crystal surface as discussed above.

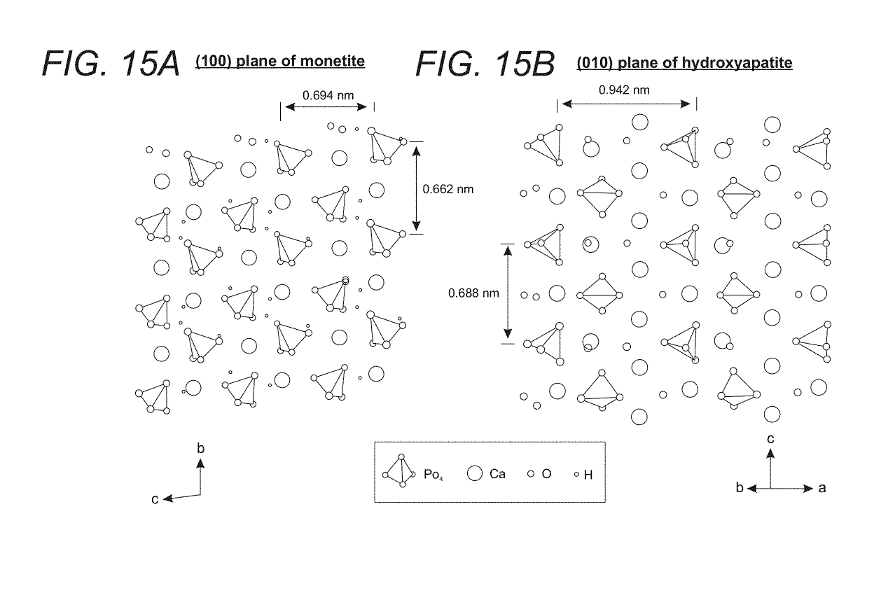

Given all these considerations, we ascribe the formation of our hierarchical HAp to a topotactic solid-solid transformation under the mediation of the chitosan-MAc matrix. The intraparticle topotactic transition was revealed by HRTEM and FFT analysis. FIG. 9D depicts a HRTEM image of a single crystal of hierarchical HAp-chitosan composite obtained from the transformation of a monetite crystal, which clearly exhibits the lattice fringes of the (002) plane of the HAp (d=0.344 nm), as well as the (020) plane of the monetite (d=0.333 nm). Furthermore, we can observe an amorphous region located at the monetite-HAp boundary (FIG. 9D), probably indicating ion rearrangement inside the crystal.⁶² Additionally, the corresponding FFT image shows two different patterns containing both HAp and monetite spots (inset in FIG. 9D). Note that the FFT spots of HAp and monetite are projected along the [010] and [100] axes, respectively, and the spots indexed as monetite (020) and HAp (002) nearly overlap, indicating that the (002) planes of HAp are nearly parallel to the (020) planes of the monetite crystals. These results suggest that the HAp crystals form on the (100) face of the CaHPO₄ single crystals and orient along the b-axis of CaHPO₄ because of the similarity of the CaHPO₄ and HAp crystal structures (FIG. 15). As a result, the (010) plane of CaHPO₄ may transform into the (001) face of HAp, as indicated by HRTEM and FFT analysis (FIG. 9d), leading to the eventual formation of HAp with bundle-like structure on the nanoscale. In addition, the chitosan-MAc matrix is stable on the crystal surface because of the low solubility of chitosan under alkaline conditions. Because of the protective effect of the chitosan-MAc matrix, the orderly layered structure of monetite-chitosan composite is inherited from the layered precursor during the topotactic phase transformation from monetite to HAp.

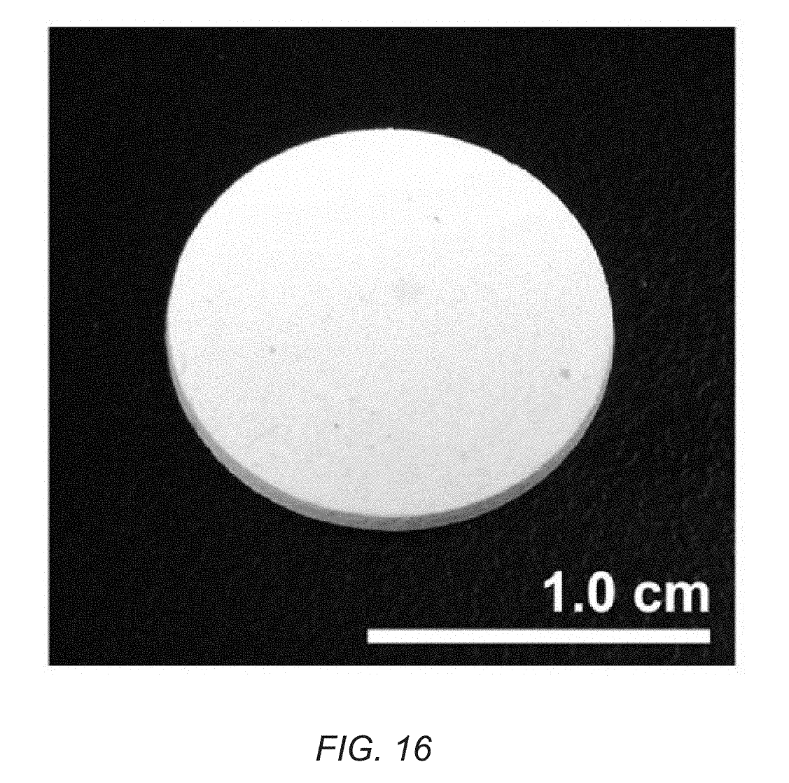

Mechanical Properties of Organized CaP-Based Composites. Because the poor mechanical properties of conventional CaP materials limit their application to non- or moderate load-bearing applications, improving their strength is of great interest. From a clinical point of view, an ideal CaP bone substitute material should have mechanical properties similar or superior to those of the bone tissue being replaced. To evaluate the mechanical properties of the synthetic CaPbased composites, we prepared bulk tablets via a simple hotpressing process using layered monetite-chitosan-MAc and organized HAp-chitosan-MAc composites as the building blocks (FIG. 16) and assessed the elastic modulus and hardness of these tablets by nanoindentation. As shown in Table 1, the elastic modulus and hardness of the tablets prepared from layered monetite-chitosan-MAc composite were measured to be 8.78±0.59 and 0.31±0.16 GPa, respectively, which are greater than those of conventional bone substitute materials reported in the literatures.^{63,64} For example, the elastic modulus of the monetite-chitosan-MAc tablet was nearly 16 times higher compared to that of conventional CaP cement (0.55±0.09 GPa).⁶⁴ By achieving a multilevel ordered structure, the elastic modulus and hardness of the tablets obtained from organized HAp-chitosan-MAc composite were significantly greater, 12.82±3.74 GPa (p<0.001) and 0.40±0.17 GPa (p<0.05), respectively. Remarkably, the elastic modulus of HAp-chitosan-MAc composite tablets was comparable to that of bone (12.7±1.7 GPa for cancellous bone and 12.9±2.2 GPa for cortical bone)⁶³ and human dentin (11.59-16.33 GPa).¹⁷ This improved mechanical performance could be explained by the extrinsic crack bridging and crack deflection in these hierarchical CaP composite materials.¹³ Specifically, the chitosan-MAc matrix, acting as viscoelastic glue, generates limited deformation between the crystals, thereby allowing the relief of locally high stresses. In addition, the hierarchical structure could toughen the CaP materials by deflecting the crack path from the plane of maximum tensile stress. The improved mechanical properties of the hierarchical CaP materials, together with their well-known biocompatibility and osteoconductivity, indicate the great potential for hard tissue engineering applications.

TABLE 1
Comparison of the Hardness and Elastic Modulus of
Reported Bone Substitute Materials, Organized CaP
Based Composites, And Natural Biological Materials^{a}

| | | hardness | elastic modulus | |
|---|---|---|---|---|
| CaP-based materials | | (GPa) | (GPa) | ref |
| Conventional | Conventional | | 0.55 ± 0.09^{b} | 63 |
| CaP materials | CaP cement | | | |
| | Fiber reinforced | | 1.03 ± 0.28^{b} | 63 |
| | CaP cement | | | |
| | Bioactive CaP | 0.24-0.27^{c} | 3.6-5.2^{c} | 64 |
| | Substitute | | | |
| Biological CaP | Human dentine | 0.52-0.91^{c} | 11.59-16.33^{c} | 15 |
| materials | Cancellous bone | 0.63 ± 0.11^{c} | 12.7 ± 1.7^{c} | 64 |
| | Cortical bone | 0.89 ± 0.16^{c} | 12.9 ± 2.2^{c} | 64 |
| Organized CaP- | Layered | 0.31 ± 0.16^{c} | 8.78 ± 0.59^{c} | |
| chitosan-Mac | monetite- | | | |
| composite | chitosan-MAc | | | |
| materials | composite tablet | | | |
| | Organized HAp- | 0.40 ± 0.17^{c},* | 12.82 ± 3.74^{c},*** | |
| | chitosan-MAc | | | |
| | composite tablet | | | |

^{a}*p < 0.05 and ***p < 0.001 compared to monetite-chitosan tablets.
^{b}Tested using an Instron testing machine.
^{c}Tested using a nanoindenter.

To determine the promise of hierarchical CaP materials as bone substitute materials, future studies are needed to optimize its structure and layers dimensions, investigate the effects of these materials on cellular functions, and study the tissue responses to them in vitro and in vivo. Another subject of further research includes the safety assessment of the ingredients within the hierarchical CaP-based composites. One of the major concerns is the toxicity of maleic acid, which has been shown to be a dermal and/or ocular irritant as a free acid.⁶⁵ A possible strategy to lower the concentration of free maleic acid is to neutralize it into various maleate salts.⁶⁵ The design of a pH-sensitive layer that can release the acid in a controlled dose is another strategy that could be used to reduce the toxicity during the degradation of hierarchical CaP-based composites.⁶⁶

### CONCLUSION

To achieve hierarchical structure in CaP materials, we devised a novel pathway to generate a layered monetite composite that can further transform into a HAp composite with a multilevel hierarchical structure. A nacre-inspired matrix composed of chitosan and maleic acid was designed to mediate the construction of an orderly layered structure. The chitosan and MAc molecules assembled into an organized complex and further guided the mineralization of monetite crystals, resulting in the formation of organized and parallel arrays of monetite platelets with a brick-and-mortar structure. By hydrolyzing the layered monetite composite in an alkali solution, hierarchical HAp with multiscale ordered structure was formed through a phase transformation under the mediation of a chitosan-MAc matrix. This hierarchical structure imparts the CaP material with improved mechanical properties and demonstrates its potential applications in hard tissue engineering. We anticipate that the approach demonstrated here will provide inspiration for biomimetic designs of advanced, mechanically robust materials for biomedical applications.

As required, detailed embodiments of the present invention are disclosed herein; however, it is to be understood that the disclosed embodiments are merely exemplary of the invention that may be embodied in various and alternative forms. The figures are not necessarily to scale; some features may be exaggerated or minimized to show details of particular components. Therefore, specific structural and functional details disclosed herein are not to be interpreted as limiting, but merely as a representative basis for teaching one skilled in the art to variously employ the present invention.

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

### REFERENCES

(1) Inzana, J. A.; Olvera, D.; Fuller, S. M.; Kelly, J. P.; Graeve, O. A.; Schwarz, E. M.; Kates, S. L.; Awad, H. A. 3D printing of composite calcium phosphate and collagen scaffolds for bone regeneration. Biomaterials 2014, 35 (13), 4026-4034.
(2) Surmenev, R. A.; Surmeneva, M. A.; Ivanova, A. A. Significance of calcium phosphate coatings for the enhancement of new bone osteogenesis-A review. ActaBiomater. 2014, 10 (2), 557-579.
(3) Ginebra, M.-P.; Canal, C.; Espanol, M.; Pastorino, D.; Montufar, E. B. Calcium phosphate cements as drug delivery materials. Adv. Drug Delivery Rev. 2012, 64 (12), 1090-1110.
(4) Akkarachaneeyakorn, K.; Li, M.; Harris, J.; Davis, S. A.; Mann, S. Bioactive hybrid organogels based on miniemulsion synthesis of morphologically complex polymer/surfactant/calcium phosphate nanostructures. Chem. Mater. 2014, 26 (20), 5965-5972.
(5) Geffers, M.; Groll, J.; Gbureck, U. Reinforcement strategies for load-bearing calcium phosphate biocements. Materials 2015, 8 (5), 2700-2717.
(6) Canal, C.; Ginebra, M. P. Fibre-reinforced calcium phosphate cements: a review. J. Mech. Behav. Biomed. Mater. 2011, 4 (8), 1658-1671.
(7) Sugawara, A.; Asaoka, K.; Ding, S.-J. Calcium phosphate-based cements: clinical needs and recent progress. J. Mater. Chem. B 2013, 1 (8), 1081-1089.
(8) Naleway, S. E.; Porter, M. M.; McKittrick, J.; Meyers, M. A. Structural design elements in biological materials: application to bioinspiration. Adv. Mater. 2015, 27 (37), 5455-5476.
(9) Zhao, N.; Wang, Z.; Cai, C.; Shen, H.; Liang, F.; Wang, D.; Wang, C.; Zhu, T.; Guo, J.; Wang, Y.; Liu, X.; Duan, C.; Wang, H.; Mao, Y.; Jia, X.; Dong, H.; Zhang, X.; Xu, J. Bioinspired materials: from low to high dimensional structure. Adv. Mater. 2014, 26 (41), 6994-7017.
(10) Mirkhalaf, M.; Dastjerdi, A. K.; Barthelat, F. Overcoming the brittleness of glass through bio-inspiration and micro-architecture. Nat. Commun. 2014, 5, 3166.
(11) Bouville, F.; Maire, E.; Meille, S.; Van de Moortele, B.; Stevenson, A. J.; Deville, S. Strong, tough and stiff bioinspired ceramics from brittle constituents. Nat. Mater. 2014, 13 (5), 508-514.
(12) Le Ferrand, H.; Bouville, F.; Niebel, T. P.; Studart, A. R. Magnetically assisted slip casting of bioinspired heterogeneous composites. Nat. Mater. 2015, 14 (11), 1172-1179.
(13) Wegst, U. G. K.; Bai, H.; Saiz, E.; Tomsia, A. P.; Ritchie, R. O. Bioinspired structural materials. Nat. Mater. 2014, 14 (1), 23-36.
(14) Ruan, Q.; Moradian-Oldak, J. Amelogenin and enamel biomimetics. J. Mater. Chem. B 2015, 3 (16), 3112-3129.
(15) Halgas, R.; Dusza, J.; Kaiferov\check{a}, J.; Ková csová, L.; Markovská, N′. Nanoindentation testing of human enamel and dentin. CeramicsSilikatý 2013, 57 (2), 92-99.
(16) Studart, A. R. Towards high-performance bioinspired composites. Adv. Mater. 2012, 24 (37), 5024-5044.
(17) Bai, H.; Walsh, F.; Gludovatz, B.; Delattre, B.; Huang, C.; Chen, Y.; Tomsia, A. P.; Ritchie, R. O. Bioinspired hydroxyapatite/poly(methyl methacrylate) composite with a nacre-mimetic architecture by a bidirectional freezing method. Adv. Mater. 2016, 28, 50.
(18) Li, C.; Born, A.-K.; Schweizer, T.; Zenobi-Wong, M.; Cerruti, M.; Mezzenga, R. Amyloid-hydroxyapatite bone biomimetic composites. Adv. Mater. 2014, 26 (20), 3207-3212.
(19) He, W.-X.; Rajasekharan, A. K.; Tehrani-Bagha, A. R.; Andersson, M. Mesoscopically ordered bone-mimetic nanocomposites. Adv. Mater. 2015, 27 (13), 2260-2264.
(20) He, W.; Kjellin, P.; Currie, F.; Handa, P.; Knee, C. S.; Bielecki, J.; Wallenberg, L. R.; Andersson, M. Formation of bone-like nanocrystalline apatite using self-assembled liquid crystals. Chem. Mater. 2012, 24 (5), 892-902.
(21) Matlahov, I.; Iline-Vul, T.; Abayev, M.; Lee, E. M. Y.; NadavTsubery, M.; Keinan-Adamsky, K.; Gray, J. J.; Goobes, G. Interfacial mineral-peptide properties of a mineral binding peptide from osteonectin and bone-like apatite. Chem. Mater. 2015, 27 (16), 5562-5569.
(22) Yang, Y.; Lv, X. P.; Shi, W.; Li, J. Y.; Li, D. X.; Zhou, X. D.; Zhang, L. L. 8DSS-promoted remineralization of initial enamel caries in vitro. J. Dent. Res. 2014, 93 (5), 520-4.
(23) Bradt, J.-H.; Mertig, M.; Teresiak, A.; Pompe, W. Biomimetic mineralization of collagen by combined fibril assembly and calcium phosphate formation. Chem. Mater. 1999, 11 (10), 2694-2701.
(24) Fan, Y.; Sun, Z.; Moradian-Oldak, J. Controlled remineralization of enamel in the presence of amelogenin and fluoride. Biomaterials 2009, 30 (4), 478-483.
(25) Ruan, Q.; Zhang, Y.; Yang, X.; Nutt, S.; Moradian-Oldak, J. An amelogenin-chitosan matrix promotes assembly of an enamel-like layer with a dense interface. ActaBiomater. 2013, 9 (7), 7289-7297.
(26) Wu, D.; Yang, J.; Li, J.; Chen, L.; Tang, B.; Chen, X.; Wu, W. Hydroxyapatite-anchored dendrimer for in situ remineralization of human tooth enamel. Biomaterials 2013, 34 (21), 5036-5047.
(27) Li, J.; Yang, J.; Li, J.; Chen, L.; Liang, K.; Wu, W.; Chen, X.; Li, J. Bioinspired intrafibrillar mineralization of human dentine by PAMAM dendrimer. Biomaterials 2013, 34 (28), 6738-47.
(28) Chen, X.; Wang, W.; Cheng, S.; Dong, B.; Li, C. Y. Mimicking bone nanostructure by combining block copolymer self-assembly and 1D crystal nucleation. ACS Nano 2013, 7 (9), 8251-8257.
(29) Hu, Y. Y.; Liu, X. P.; Ma, X.; Rawal, A.; Prozorov, T.; Akinc, M.; Mallapragada, S. K.; Schmidt-Rohr, K. Biomimetic self-assembling copolymer-hydroxyapatite nanocomposites with the nanocrystal size controlled by citrate. Chem. Mater. 2011, 23 (9), 2481-2490.
(30) Li, L.; Mao, C.; Wang, J.; Xu, X.; Pan, H.; Deng, Y.; Gu, X.; Tang, R. Bio-inspired enamel repair via Glu-directed assembly of apatite nanoparticles: an approach to biomaterials with optimal characteristics. Adv. Mater. 2011, 23 (40), 4695-4701.
(31) Busch, S. Regeneration of human tooth enamel. Angew. Chem., Int. Ed. 2004, 43 (11), 1428-1431.
(32) Furuichi, K.; Oaki, Y.; Imai, H. Preparation of nanotextured and nanofibrous hydroxyapatite through dicalcium phosphate with gelatin. Chem. Mater. 2006, 18 (1), 229-234.
(33) Ito, H.; Oaki, Y.; Imai, H. Selective synthesis of various nanoscale morphologies of hydroxyapatite via an intermediate phase. Cryst. Growth Des. 2008, 8 (3), 1055-1059.
(34) Zou, Z.; Liu, X.; Chen, L.; Lin, K.; Chang, J. Dental enamel-like hydroxyapatite transformed directly from monetite. J. Mater. Chem. 2012, 22 (42), 22637-22641.
(35) Liu, X.; Lin, K.; Wu, C.; Wang, Y.; Zou, Z.; Chang, J. Multilevel hierarchically ordered artificial biomineral. Small 2014, 10 (1), 152-9. (36) Addadi, L.; Joester, D.; Nudelman, F.; Weiner, S. Mollusk Shell Formation: A source of new concepts for understanding biomineralization processes. Chem.—Eur. J. 2006, 12 (4), 980-987.
(36) Addadi, L.; Joester, D.; Nudelman, F.; Weiner, S. Mollusk Shell Formation: A source of new concepts for understanding biomieralization processes. Chem.—Eur. J. 2006, 12 (4), 980-987
(37) Levi-Kalisman, Y.; Falini, G.; Addadi, L.; Weiner, S. Structure of the nacreous organic matrix of a bivalve mollusk shell examined in the hydrated state using cryo-TEM. J. Struct. Biol. 2001, 135 (1), 8-17.
(38) Suzuki, M.; Saruwatari, K.; Kogure, T.; Yamamoto, Y.; Nishimura, T.; Kato, T.; Nagasawa, H. An acidic matrix protein, Pif, is a key macromolecule for nacre formation. Science 2009, 325 (5946), 1388-90.
(39) Baskar, D.; Balu, R.; Kumar, T. S. S. Mineralization of pristine chitosan film through biomimetic process. Int. J. Biol. Macromol. 2011, 49 (3), 385-389.
(40) He, L.-H.; Yao, L.; Xue, R.; Sun, J.; Song, R. In-situ mineralization of chitosan/calcium phosphate composite and the effect of solvent on the structure. Front. Mater. Sci. 2011, 5 (3), 282-292.
(41) Li, L.; Zhao, M.; Ding, S.; Zhou, C. Rapid biomimetic mineralization of chitosan scaffolds with a precursor sacrificed method in ethanol/water mixed solution. eXPRESSPolym. Lett. 2011, 5 (6), 545-554.
(42) Jin, J.; Hassanzadeh, P.; Perotto, G.; Sun, W.; Brenckle, M. A.; Kaplan, D.; Omenetto, F. G.; Rolandi, M. A biomimetic composite from solution self-assembly of chitin nanofibers in a silk fibroin matrix. Adv. Mater. 2013, 25 (32), 4482-4487.
(43) Arias, J. L.; Fernandez, M. S. Biomimetic processes through the study of mineralized shells. Mater. Charact. 2003, 50 (2-3), 189-195.
(44) Sailakshmi, G.; Mitra, T.; Chatterjee, S.; Gnanamani, A. Engineering chitosan using α, ω-dicarboxylic acids—an approach to improve the mechanical strength and thermal stability. J. Biomater. Nanobiotechnol. 2013, 4 (2), 151-164.
(45) Mitra, T.; Sailakshmi, G.; Gnanamani, A.; Mandal, A. B. Studies on cross-linking of succinic acid with chitosan/collagen. Mater. Res. 2013, 16, 755-765.
(46) Liu, W.; Sun, S.; Cao, Z.; Zhang, X.; Yao, K.; Lu, W. W.; Luk, K. D. An investigation on the physicochemical properties of chitosan/DNA polyelectrolyte complexes. Biomaterials 2005, 26 (15), 2705-11.
(47) Tan, Z. H.; Jin, S. H.; Li, N. Basic Organic Chemistry; Scientific and Technical Documents Publishing House: Beijing, 2004.
(48) Domard, A. Determination of N-acetyl content in chitosan samples by c.d. measurements. Int. J. Biol. Macromol. 1987, 9 (6), 333-336.
(49) Feng, W.; Li, Y.; Ji, P. Interaction of water soluble chitosan with multiwalled carbon nanotubes. AIChE J. 2012, 58 (1), 285-291.
(50) Li, W.; Liu, Y.; Perez, T.; Gunton, J. D.; Sorensen, C. M.; Chakrabarti, A. Kinetics of nanochain formation in a simplified model of amelogenin biomacromolecules. Biophys. J. 2011, 101 (10), 2502-2506.
(51) Li, W.; Chakrabarti, A.; Gunton, J. D. Self-assembly of a bipolar model of biomacromolecules. Langmuir 2013, 29 (14), 4470-4476.
(52) Assis, O. B. G.; Bernardes-Filho, R.; Vieira, D. C.; Filho, S. P. C. AFM characterization of chitosan self-assembled films. Int. J. Polym. Mater. 2002, 51 (7), 633-638.
(53) Wang, Z.; Zeng, R.; Tu, M.; Zhao, J. Synthesis, characterization of biomimetic phosphorylcholine-bound chitosan derivative and in vitro drug release of their nanoparticles. J. Appl. Polym. Sci. 2013, 128 (1), 153-160.
(54) Liu, J.; Zhang, L.; Wang, C.; Xu, H.; Zhao, X. Preparation and characterization of lectin-conjugated chitosan fluorescent nanoparticles. Mol. BioSyst. 2010, 6 (6), 954-7.
(55) Deemak, P.; Wanichwecharungruang, S.; Nonthabenjawan, R.; Jornjangjun, C. Controlling the morphology of self-assemble chitosan through derivatization. J. Polym. Res. 2011, 18 (3), 419-424.
(56) Yang, M.; Chen, G.; Zhao, Y.; Silber, G.; Wang, Y.; Xing, S.; Han, Y.; Chen, H. Mechanistic investigation into the spontaneous linear assembly of gold nanospheres. Phys. Chem. Chem. Phys. 2010, 12 (38), 11850-11860.
(57) Zhang, H.; Fung, K.-H.; Hartmann, J.; Chan, C. T.; Wang, D. Controlled chainlike agglomeration of charged gold nanoparticles via a deliberate interaction balance. J. Phys. Chem. C 2008, 112 (43), 16830-16839.
(58) Li, W.; Kanyo, I.; Kuo, C.-H.; Thanneeru, S.; He, J. pHprogrammable self-assembly of plasmonic nanoparticles: hydrophobic interaction versus electrostatic repulsion. Nanoscale 2015, 7 (3), 956-964.
(59) Max, J.-J.; Chapados, C. Infrared spectroscopy of aqueous carboxylic acids: malic acid. J. Phys. Chem. A 2002, 106 (27), 6452-6461.
(60) Dosen, A.; Giese, R. F. Thermal decomposition of brushite, CaHPO₄2H₂O to monetite CaHPO₄ and the formation of an amorphous phase. Am. Mineral. 2011, 96 (2-3), 368-373.
(61) Xiao, J.; Zhu, Y.; Ruan, Q.; Liu, Y.; Zeng, Y.; Xu, F.; Zhang, L. Biomacromolecule and surfactant complex matrix for oriented stack of 2-dimensional carbonated hydroxyapatite nanosheets as alignment in calcified tissues. Cryst. Growth Des. 2010, 10 (4), 1492-1499.
(62) Wang, M.; Gao, J.; Shi, C.; Zhu, Y.; Zeng, Y.; Wang, D. Facile one-pot synthesis of oriented pure hydroxyapatite with hierarchical architecture by topotactic conversion. Cryst. Growth Des. 2014, 14 (12), 6459-6466.
(63) Panzavolta, S.; Bracci, B.; Focarete, M. L.; Gualandi, C.; Bigi, A. Fiber reinforcement of a biomimetic bone cement. J. Mater. Sci.: Mater. Med. 2012, 23 (6), 1363-1370.
(64) Ni, G. X.; Choy, Y. S.; Lu, W. W.; Ngan, A. H. W.; Chiu, K. Y.; Li, Z. Y.; Tang, B.; Luk, K. D. K. Nano-mechanics of bone and bioactive bone cement interfaces in a load-bearing model. Biomaterials 2006, 27 (9), 1963-1970.
(65) Cosmetic Ingredient Review Expert Panel. Final report on the safety assessment of Maleic Acid. Int. J. Toxicol. 2007, 26 (S2), 125-130.
(66) Sen, M.; Uzun, C.; Guven, O. Controlled release of terbinafine hydrochloride from pH sensitive poly(acrylamide/maleic acid) hydrogels. Int. J. Pharm. 2000, 203 (1-2), 149-157.

What is claimed is:

1. A method for forming a composite material comprising:
a) dissolving chitosan, a dicarboxylic acid, NaH₂PO₄ and CaCl₂.2H₂O in water to form a reaction solution;
b) heating the reaction solution to form a layered monetite/chitosan/dicarboxylic acid composite; and
c) transforming the layered monetite/chitosan/dicarboxylic acid composite to a calcium apatite/chitosan/dicarboxylic acid composite.

2. The method of claim 1 wherein the calcium apatite/chitosan/dicarboxylic acid composite is formed by contacting the layered monetite/chitosan/dicarboxylic acid composite with base to form the calcium apatite/chitosan/dicarboxylic acid composite.

3. The method of claim 1 wherein the dicarboxylic acid is selected from the group consisting of maleic acid, oxalic acid, fumaric acid, and combinations thereof.

4. The method of claim 1 wherein the dicarboxylic acid is maleic acid.

5. The method of claim 1 wherein the calcium apatite/chitosan/dicarboxylic acid composite is a hydroxylapatite/chitosan/dicarboxylic acid composite.

6. The method of claim 5 wherein the calcium apatite/chitosan/dicarboxylic acid composite is a hydroxylapatite/chitosan/maleic acid composite.

7. The method of claim 5 wherein the hydroxylapatite/chitosan/dicarboxylic acid composite is a multilayered structure having at least 3 organic matrix layers and at least 3 calcium apatite-containing layers.

8. The method of claim 7 having at least 5 organic matrix layers and at least 5 calcium phosphate-containing layers.

9. The method of claim 7 having from 3 to 30 organic matrix layers and from 3 to 30 calcium phosphate-containing layers.

10. The method of claim 7 wherein the organic matrix layers include the chitosan and the dicarboxylic acid.

11. The method of claim 7 wherein the chitosan in the hydroxylapatite/chitosan/dicarboxylic acid composite is cross-linked by the dicarboxylic acid.

12. The method of claim 7 wherein the organic matrix layers have an average thickness from about 50 nm to about 1 micron.

13. The method of claim 7 wherein the organic matrix layers have an average thickness from about 50 nm to about 200 nm.

14. A multilayer structure that functions as a bone replacement material, the multilayer structure comprising:
a plurality of organic matrix layers, the organic matrix layer including chitosan and a dicarboxylic acid; and
a plurality of calcium phosphate-containing layers wherein each calcium phosphate-containing layer is interposed between a pair of organic matrix layers.

15. The multilayer structure of claim 14 wherein the chitosan is cross-linked by the dicarboxylic acid.

16. The multilayer structure of claim 14 wherein the dicarboxylic acid is maleic acid, oxalic acid, or fumaric acid.

17. The multilayer structure of claim 14 wherein the calcium phosphate-containing layers are hydroxyapatite layers or monetite layers.

18. The multilayer structure of claim 14 wherein the organic matrix layers have an average thickness from about 50 nm to about 1 micron.

19. The multilayer structure of claim 14 wherein the organic matrix layers have an average thickness from about 50 nm to about 200 nm.

20. The multilayer structure of claim 14 wherein the calcium phosphate-containing layers have an average thickness from about 50 nm to about 1 micron.

21. The multilayer structure of claim 14 wherein the calcium phosphate-containing layers have an average thickness from about 50 nm to about 200 nm.

22. The multilayer structure of claim 14 having at least 3 organic matrix layers and at least 3 calcium phosphate-containing layers.

23. The multilayer structure of claim 14 having at least 5 organic matrix layers and at least 5 calcium phosphate-containing layers.

24. The multilayer structure of claim 14 having from 3 to 30 organic matrix layers and from 3 to 30 calcium phosphate-containing layers.
